(19) United States
(12) Patent Application Publication
(10) Pub. No.: US 2024/0254175 A1
(43) Pub. Date: Aug. 1, 2024

(54) NOVEL SELECTIVE ANTIMICROBIAL FUSION PEPTIDES

(71) Applicant: BOMBI BIOMICS BV, Eindhoven (NL)

(72) Inventor: Jeroen HOFENK, Eindhoven (NL)

(21) Appl. No.: 18/263,431

(22) PCT Filed: Jan. 31, 2022
(86) PCT No.: PCT/EP2022/052193

§ 371 Date: Jul. 28, 2023

(30) Foreign Application Priority Data
Jan. 29, 2021 (EP) 21154190.9

(51) Int. Cl. C07K 14/46; A61K 9/00; A61K 38/00; A61K 47/06; A61K 47/08; A61K 47/10; A61K 47/18; A61K 47/26; A61K 47/32; A61K 47/36; A61K 47/44; A61P 31/04

(52) U.S. Cl. CPC C07K 14/463; A61K 9/00; A61K 47/06; A61K 47/08; A61K 47/10; A61K 47/18; A61K 47/26; A61K 47/32; A61K 47/36; A61K 47/44; A61P 31/04; A61K 38/00

(57) ABSTRACT

Antimicrobial fusion peptides include a polypeptide having at least 35 amino acids and containing the amino acid sequence GIGGALLSAG X¹SALX²GLAX³G LAEHFAN, where X¹, X², and X³ independently represent lysine, glutamic acid, or glutamine, and wherein at least one of X¹, X², or X³ is independently selected from glutamic acid or glutamine, including variants with at least 97% sequence homology. Pharmaceutical compositions may include the polypeptides and may be used for treatment of bacterial infection caused by C. acnes. The polypeptides are nonhemolytic and exhibit reduced in vitro cytotoxicity relative to other antimicrobial peptides, which makes them useful in pharmaceutical, healthcare, medical device, food, and personal care applications.

### CROSS-REFERENCES TO RELATED APPLICATIONS

This application is a national-stage application under 35 U.S.C. § 371 of International Application No. PCT/EP2022/052193, filed Jan. 31, 2022, which International Application claims benefit of priority to European Patent Application No. 21154190.9, filed Jan. 29, 2021.

### TECHNICAL FIELD

The present invention relates to novel antimicrobial fusion peptides; pharmaceutical compositions comprising the peptides and to uses thereof for treatment or prevention of bacterial infection caused by C. acnes. The peptides of the present invention are nonhemolytic, exhibit reduced in vitro cytotoxicity relative to other antimicrobial peptides, which makes them useful in pharmaceutical, healthcare, medical device, food, and personal care applications.

### BACKGROUND

Antimicrobial peptides (AMPs) are ubiquitous in nature and are a quintessential component in the innate immune system of many organisms throughout nature and offer a first line protection against invading pathogens [1, 2].

They are diverse in structure, function, and specificity, and usually do not target single defined molecular structures (epitopes), but rather act on the cell membrane mostly by compromising the bacterial membrane integrity. Interaction of AMPs with the anionic membrane surface of the target microbes leads to membrane permeabilization, cell lysis and death. It is generally accepted that the cytoplasmic membrane is the main target of most antimicrobial peptides, whereby accumulation of peptide in the membrane causes increased permeability and loss of barrier function, resulting in leakage of cytoplasmic components and cell death. Various molecular mechanisms for membrane permeabilization, some phenomenological and others more quantitative, have been proposed to explain the action of AMPs. Therefore, as opposed to conventional antibiotics which often have simple targets, such as a unique epitope on the cell wall, or in the protein and RNA synthesis processes, allowing the pathogenic bacteria resistance more rapidly, AMPs are effective regardless of the metabolic activity of bacteria [3, 4].

Furthermore, owing to the very fast killing rates of AMPs; being faster that the actual growth rate of bacteria, and the nature of the target, development of resistance is very less likely. In fact, the emergence of mutant strains being resistant to AMPs has been determined by monitoring bacterial susceptibility after repeated sub-culturing in the presence of sub-inhibitory concentrations of the peptides, showing that the mutation rates were lower than conventional antibiotics tested (e.g. ciprofloxacin, and erythromycin). While the Minimal Inhibiting Concentration (MIC) of those antibiotics increased through all the subcultures (up to 64 times), the pressure of the peptides did not increase the MIC of the strain. Thus, as opposed to conventional antibiotic, resistance development in the presence of AMPs is less unlikely to occur. More importantly, AMPs are also fast-acting and biodegradable, which in turn alleviates the current concerns about residual antibiotics in the environment.

The biological activity of these peptides range from being broad spectrum (active against both Gram positive, and Gram negative bacteria) and non-hemolytic (e.g., bombinin, magainin, macropin), to broad spectrum and highly hemolytic (e.g. melittin, maximins). For commercial applications of AMPs, increasing the bactericidal activity and decreasing the hemolytic activity is desirable.

Additionally to the direct antimicrobial activities of AMPs, certain of these peptides can be particularly attractive as they exhibit several other activities such as the regulation of the innate and adaptive immune system [5, 6], decrease of inflammatory responses [7, 8], stimulation of wound healing [9], additional antifungal [10], antiparasitic [11, 12], and anticancer activities [13]. As such they are particularly well suited for therapeutic purposes.

Another disadvantage of conventional antibiotics, as opposed to antimicrobial peptides, is that they do not ensure eradication of biofilm infections which are commonly associated with microbial infections. In biofilms, microorganisms aggregate in a tightly structured community and in a selfexcreted polymeric matric by which they adhere to a surface. This disadvantage can be explained by the following reasons:
1. Most antibiotics are able to penetrate biofilms, but their diffusion is slow in which they are inactivated before they can even elicit their desired effect [14, 15].
2. Low metabolic activity of bacteria in biofilm rendering antibiotics ineffective [16].
3. Degradation or inactivation of antibiotics is a common problem as they are prone to be removed from the bloodstream by renal clearance and from there on are degraded enzymatically in the blood and surrounding tissues. Even through topical application, enzymes produced by bacteria can directly destruct or modify the compound. These mechanisms actively reduce their concentration and effectiveness. In biofilms, the low penetration potential poses additional problems. As to such, increasing the administered concentration is not feasible due to the toxicity of high blood concentrations of antibiotics [17].
4. Repetitive administration of conventional antibiotics promoted the development of antibiotic resistance, in which bacterial escape from antibiotic pressure is also higher and may lead to the survival of mutants that have an increased resistance to these antibiotics [18, 19].
5. Conventional antibiotics are known to be responsible for the release of pro-inflammatory microbial compounds, known as “Pathogen-Associated Molecular Patterns” (PAMPs). In the case of infection, the host immune system is additionally triggered by these molecules which are recognized by specific receptors on the host cells, such as Toll-like Receptors (TLRs). For example, bacterial peptidoglycan and/or lipopolysaccharide are recognized by TLR2 and TLR4, thereby potently inducing an inflammatory cascade of events. This activation of the immune system, both by the foreign body response and the bacterial infection, leads to an extrapolated reaction of they host immune system, ultimately leading to inflamed and disrupted tissue which in fact, in turn, provides the ideal environment for infection [20].

At present, according to the Data Repository of Antimicrobial Peptides (DRAMP) database (dramp.cpu-bioinfor.org), over 5,780 different antimicrobial peptide sequences are known and the database contains more than 20,500 entries of which more than 14,700 are patent related AMPs, and 76 AMPs are in drug development (either preclinical or clinical stage).

As opposed to broad-spectrum AMPs, there are also several naturally occurring AMPs, including plantaricin, lantibiotic, and nisin, that were found to contain both targeting and antimicrobial domains within their peptide sequences [21, 22, 23]. The presence of targeting domains promoted the accumulation of peptides on the bacterial membrane, increasing local peptide concentrations and enhancing bactericidal activities against the targeted microbes [24, 25].

Furthermore, several attempts have been made in recent years, to construct species-specific antimicrobials by conjugating a bacterial recognition domain to antimicrobial peptides or bactericidal proteins [26, 27, 28, 29, 30, 31].

These findings provided an intriguing starting point for the development of multidomain AMPs with selective bactericidal effects against specific bacteria. Although all of these synthetic molecules exhibited enhanced antimicrobial potency, selectivity, and kinetics against specific bacteria, inevitable bactericidal effects on unrelated bacteria were also observed with these peptides. The main reason for this problem may lay in the fact that the killing moiety of these fusion peptides had not been redesigned or modified, and thus electrostatic attractions between cationic peptide molecules and anionic lipids of the bacterial membrane were retained, thereby causing in-discriminant bactericidal effect via a non-receptor-mediated pathway. Thus, these approaches still have room for improvement and have yet to generate target-specific antimicrobials.

Therefore, even though there are a relatively large number of antimicrobial peptides available today, there is still an increased need of new improved antimicrobial peptides, which can be used to counteract microbes, in particular, those that are resistant or tolerant against antibiotic agents and/or other antimicrobial agents. More importantly, there is a need for new antimicrobial peptides, which are non-allergenic when introduced into mammals such as human beings and that have high species specificity against pathogenic microorganisms.

### SUMMARY

The present invention provides novel species-targeted antimicrobial peptides (STAMPs) that can be designed and improved via a tunable “building-block” approach. The peptides overcome the shortcomings of conventional antibiotics, have improved properties over known broad-spectrum antimicrobial peptides, and exert high selectivity and activity against specific pathogenic microorganisms in biofilm infections.

The present invention provides novel, selective antibacterial agents that are based on the addition of a bacteria species-specific quorum sensing peptide motif derived from its S-ribosylhomocysteine lyase gene to an existing broad-spectrum AMP, known as Bombinin-like peptide-7 (BLP-7), derived from Bombina orientalis (Oriental fire-bellied toad), and thereby generating STAMPs that are selective for a particular bacterial species or strain; a feature that is not made possible by the otherwise broadspectrum native BLP-7.

Said completed STAMPs consist of conjoined but functionally independent targeting and killing regions, separated by a small linker. All within said linear peptides, the N-terminus is preferably further functionalized by palmitoylation and the C-terminus is preferably functionalized by amidation.

A particular advantage of the present invention is that said STAMPs targeting regions drive enhancement of antimicrobial activity by increasing binding to the surface of a targeted pathogen, utilizing specific determinants such as overall membrane hydrophobicity, charge, and/or quorum sensing motifs, which in turn leads to increased selective accumulation of the killing moiety.

Another particular advantage of the present invention is that said STAMPs active killing region can be optimized by weakening the electrostatic attractions between the peptide and the anionic lipids of the bacterial membrane and thereby preventing the molecules from entering the microbial membrane, decreasing or abolishing antimicrobial activity against untargeted bacterial species, while maintaining potent antimicrobial activity towards the quorum-sensing motifs of specific bacteria.

In a particular embodiment, a further advantage of the present invention is to significantly reducing the risk of proteolytic degradation of said STAMPs by synthesizing them using D-form peptides instead of L-form peptides.

As compared to its relative L-form, it has been found that such D-form peptides also increases antimicrobial activity via specific association with bacterial cell wall components, including lipoteichoic acid (LTA), and peptidoglycan (PG).

In another particular embodiment, it has been found in the present invention that the fusion peptide BMBN-7, having the sequence

(SEQ ID NO: 8)
ERNNF-GGG-GIGGALLSAGESALKGLAKGLAEHFAN

in which the fusion peptide is synthesized using D-form peptides instead of L-form peptides, is highly effective against gram-positive Cutibacterium acnes (C. acnes) without showing the ability to inhibit the growth of other microbial species in vitro.

As shown in (FIGS. 2A and 2B), BMBN-7 is considerably more effective than any other peptide tested, and is equally effective in killing C. acnes as compared to its wildtype counterpart BLP7. Moreover, BMBN-7 neutralizes endotoxins LTA and PG, thus reducing the proinflammatory response while at the same time, showing only negligible hemolytic activity against human erythrocytes. BMBN-7 adapts cationic amphipathic α-helical structural features of BLP7 in which polar amino acids are located at one side of the helix and lipophilic amino acids at the opposite side.

At position 11 of the wildtype BPL7, amino acid K (lysine) was substituted with the neutral amino acid E (glutamic acid) in order to lower the net positive charge from 2.8 to 0.9 at pH 5.5 which weakened the electrostatic attraction between the active killing moiety of BMBN-7 and the anionic lipids of C. acnes membrane. This reduction resulted in the loss of bactericidal effects towards all the untargeted bacteria while maintaining robust antimicrobial activity against C. acnes: BMBN-7 exhibited a minimum inhibitory concentration (MIC) against C. acnes 7 times lower than BLP7 alone, indicating a unique specificity for the targeted bacteria.

Furthermore, the substitution of the bacteria species-specific quorum sensing peptide motif, having the sequence of ERNNF (FIG. 1) of BMBN-7 with a random peptide sequence (EFGGQ) led to the loss of antimicrobial activity against C. acnes (FIG. 3A). These results indicate that the specificity of BMBN-7 against the targeted bacteria is dependent on the bacteria species-specific quorum sensing peptide motif interactions.

As is shown in (FIG. 3B) and detailed in the examples, BMBN-7 has an IC99.9 (3.22 μM) that is even lower than the IC90 of wildtype BLP7 (5.13 μM). Thus, at a concentration of 3.22 μM, BMBN-7 kills 999 out of 1000 bacteria, whereas wildtype BLP7 kills only 900 out of 1000 bacteria at a higher concentration. Thus, 100 times more bacteria survive after treatment with wildtype BLP7 as compared to treatment with BMBN-7 at a similar concentration.

Accordingly, in one embodiment the disclosure of the present invention, comprises the following amino acid sequence ERNNF-GGG-GIGGALLSAGESALKGLAKGLAEHFAN (SEQ ID NO:8/FIG. 1), or a variant of the amino acid sequences with at least 85%, 90%, 91%, 92%, 93%, 94%, 95%, 96%, 97%, 98% or at least 99% sequence homology, said polypeptide has species-specific antimicrobial, antibacterial and/or anti-inflammatory activity.

In an embodiment according to the invention the polypeptide or variant of the amino acid sequence with at least 85%, 90%, 91%, 92%, 93%, 94%, 95%, 96%, 97%, 98% or at least 99% sequence homology is characterized in having at least 35 amino acids comprising

(SEQ ID NO: 9)
GIGGALLSAG X¹SALX²GLAX³G LAEHFAN

Wherein X¹, X² and X³ represents K (lysine); E (glutamic acid), or Q (glutamine) and wherein at least one of X¹, X² and X³ is independently selected from E (glutamic acid), or Q (glutamine).

In another embodiment at least at least two of X¹, X² and X³ are independently selected from E (glutamic acid), or Q (glutamine); and in a particular embodiment all three of X¹, X² and X³ are independently selected from E (glutamic acid), or Q (glutamine).

Expressed differently, in one embodiment of the invention the species-specific antimicrobial, antibacterial and/or anti-inflammatory polypeptides have at least 35 amino acids comprising the wildtype BLP7 sequence

(SEQ ID NO: 1)
GIGGALLSAG KSALKGLAKG LAEHFAN

Wherein up to three and at least one of the lysine's (K) at positions 11, 15 and 19 are each independently substituted with amino acid E (glutamic acid), or Q (glutamine).

In one embodiment the variant comprises a polypeptide with a substitution of position 11; amino acid K (lysine) with amino acid E (glutamic acid), or Q (glutamine) as shown hereunder:

GIGGALLSAGESALKGLAKGLAEHFAN
Or
GIGGALLSAGQSALKGLAKGLAEHFAN

In one embodiment the variant comprises a polypeptide with a substitution of position 11 and 15; amino acid K (lysine) with amino acid E (glutamic acid), or Q (glutamine) as shown hereunder:

GIGGALLSAGESALEGLAKGLAEHFAN
Or
GIGGALLSAGQSALQGLAKGLAEHFAN

In one embodiment the variant comprises a polypeptide with a substitution of position 11, 15, and 19; amino acid K (lysine) with amino acid E (glutamic acid), or Q (glutamine) as shown hereunder:

GIGGALLSAGESALEGLAEGLAEHFAN
Or
GIGGALLSAGQSALQGLAQGLAEHFAN

Substitutions can be made interchangeably choosing from either E or Q within these positions so to enable different combinations. These changes are also within the scope of the present invention and represent variants of the species-specific antimicrobial, antibacterial and/or anti-inflammatory polypeptides according to the invention

Said polypeptide or “variants” thereof as herein defined having one or more substitutions of an amino acid by a corresponding D-amino acid, having one or more substitutions of an amino acid by a corresponding non-natural amino acid, and/or having a retro-inverso sequence of at least 35 consecutive amino acids from the amino acid sequence are also within the scope of the present invention.

Accordingly, this disclosure of the present invention, also comprises substitutions of the bacteria species-specific quorum sensing peptide motif in which sequence ERNNF can also be substituted with mutant sequences: ERNNT or ERNNY.

In amino acid sequences or “variants” thereof as defined herein, amino acids are denoted by single-letter symbols. These single-letter symbols and three-letter symbols are well known to the person skilled in the art and have the following meaning: A (Ala) is alanine, C (Cys) is cysteine, D (Asp) is aspartic acid, E (Glu) is glutamic acid, F (Phe) is phenylalanine, G (Gly) is glycine, H (His) is histidine, I (Ile) is isoleucine, K (Lys) is lysine, L (Leu) is leucine, M (Met) is methionine, N (Asn) is asparagine, P (Pro) is proline, Q (Gln) is glutamine, R (Arg) is arginine, S (Ser) is serine, T (Thr) is threonine, V (Val) is valine, W (Trp) is tryptophan, and Y (Tyr) is tyrosine.

The polypeptides of the present invention have species-specific antimicrobial activity, more preferably antibacterial activity. Furthermore, the polypeptides of the present invention, preferably has both antimicrobial and anti-inflammatory activity. The term “antimicrobial activity” of a polypeptide as used herein refers to counteracting growth or proliferation of a bacterium, and includes inhibition, reduction or prevention of growth or proliferation as well as killing of the bacterium. A bacterium is an organism that is microscopic, i.e., usually too small to be seen by the naked human eye. Similarly, the term “antibacterial activity” as used herein refers to counteracting growth or proliferation of, respectively, a bacterium in general, and includes inhibition, reduction or prevention of growth or proliferation as well as killing thereof. Antimicrobial, and antibacterial activity can be measured by methods known in the art.

The term “anti-inflammatory activity” of the polypeptides as used herein refers to inhibiting, reducing or preventing an inflammatory response in a subject that has been infected by microbes, e.g., bacteria. Anti-inflammatory activity of polypeptides of the disclosure is achieved by inhibiting, reducing or preventing the release of pro-inflammatory microbial compounds, such as LTA, or PG. Antiinflammatory activity can be measured by methods known in the art. One such example is the inhibition of cytokines in RAW264.7 cells in which cells were pretreated with the polypeptides of the present invention along with treatment with PG. In this method, the polypeptides of the disclosure are mixed with 1 mg of PG as a control or infected with a C. acnes (strain HL045PA1) at a multiplicity of infection of 10 and incubated for 1 hour. Thereafter, these mixtures were added to 4 times diluted fresh human blood and 18 hours thereafter, the level of cytokines Interleukins (IL-8, IL-12) and Tumor Necrosis Factor Alpha (TNF-α) in the blood sample are measured by ELISA.

In at least one embodiment of the present invention, the polypeptides or “variants” thereof as herein defined according to the disclosure having an N-terminal and/or C-terminal modification are also within the scope of the present invention, preferably comprising an N- and/or C-terminal elongating group, the N-terminal modification preferably selected from the group consisting of an acetyl-, hexanoyl-, decanoyl-, myristoyl-, NH—(CH₂—CH₂—O)₁₁—CO— and propionyl residue, preferably N-palmitoylation and the C-terminal modification preferably selected from the group consisting of amide-, NH—(CH₂—CH₂—O)₁₁—CO-amide- and one or two amino-hexanoyl groups, preferably CONH₂. In a particular aspect of the invention the polypeptides of the present invention have an N-terminal and C-terminal modification are also within the scope of the present invention, preferably comprising an N- and C-terminal elongating group, the N-terminal modification preferably selected from the group consisting of an acetyl-, hexanoyl-, decanoyl-, myristoyl-, NH—(CH₂—CH₂—O)₁₁—CO— and propionyl residue, preferably N-palmitoylation and the C-terminal modification preferably selected from the group consisting of amide-, NH—(CH₂—CH₂—O)₁₁—CO-amide- and one or two amino-hexanoyl groups, preferably CONH₂. It will be clear to a person skilled in the art that other N- or C-terminal elongating groups will also yield active compounds.

Alternatively, or in addition to the up to 3 substitutions of an amino acid by another amino acid as described above, a “variant” of the polypeptide comprising amino acid sequence GIGGALLSAG X¹SALX²GLAX³G LAEHFAN (SEQ ID NO:9), as defined herein may contain one or more substitutions of an L-amino acid by its corresponding D-amino acid.

Amino acids indicated herein by an upper case single-letter symbol, such as S for serine, are those L-amino acids commonly found in naturally occurring proteins. Therefore, a variant of the aforementioned amino acid sequences as defined herein may contain one or more substitutions of an amino acid by the corresponding D-amino acid, preferably all L-amino acids are replaced with corresponding D-amino acids. “Corresponding D-amino acid” as used herein is defined as the D-amino acid counterpart of an L-amino acid. For example, the corresponding D-amino acid of alanine (A) is D-alanine (a), the corresponding D-amino acid of arginine (R) is D-arginine (r), the corresponding D-amino acid of asparagine (N) is D-asparagine (n), etc.

All L-amino acids of a variant as defined herein can be substituted by their corresponding D-amino acids. A variant of amino acid sequence as defined in (FIG. 1) may contain substitutions of an L-amino acid by its corresponding D-amino acid. Hence, the variant may consist entirely of D-amino acids because antimicrobial activity is retained in polypeptides comprising such amino acid variant.

A “variant” as defined herein may further contain the retro-inverso peptide of the 35 consecutive amino acids of the aforementioned amino acid sequences described herein. Preferably, the variant is a retro-inverso peptide of the full length of the amino acid sequence.

A retro-inverso peptide is a peptide consisting of D-amino acids in the reversed sequence of a reference amino acid sequence. Hence, an example of a preferred variant of the disclosure may have 35 amino acids of the D-amino acid sequence

ERNNFGGGGIGGALLSAGESALKGLAKGLAEHFAN

in which a polypeptide comprising the sequence

NAFHEALGKALGKLASEGASLLAGGIGGGGFNNRE

is a variant containing the retro-inverso sequence of amino acid sequence

(SEQ ID NO: 4/FIG.1)
ERNNFGGGGIGGALLSAGESALKGLAKGLAEHFAN.

It's an advantage of the present invention, that in a “variant” of the polypeptide comprising amino acid sequence GIGGALLSAG X¹SALX²GLAX³G LAEHFAN (SEQ ID NO:9), as defined herein one or more natural amino acids of the sequences described herein can be substituted by a corresponding non-natural amino acid.

A “corresponding nonnatural amino acid” refers to a non-natural amino acid that is a derivative of the reference natural amino acid. For instance, a natural amino acid is substituted by the corresponding β-amino acid. β-amino acids have their amino group bonded to the β carbon rather than the a carbon as in the natural amino acids. For instance, α-alanine is substituted by β-alanine, etc. Other examples of substitution of a natural amino acid by a non-natural amino acid that is a derivative of the natural amino acid are the following. Alanine is, for instance, substituted by beta-alanine, t-butylalanine, 2-napthylalanine, L-3-(2-naphthyl)alanine, and 2-aminoisobutyric acid. Arginine is, for instance, substituted by homoarginine, ornithine, N5-carbamoylornithine, and 3-amino-propionic acid. Asparagine is, for instance, substituted by N-ethylasparagine. Aspartic acid is, for instance, substituted by 4-tert-butyl hydrogen 2-azidosuccinate. Cysteine is, for instance, substituted by cysteic acid and homocysteine. Glutamic acid is, for instance, substituted by γ-carboxy-DL-glutamic acid and 4-fluoro-DL-glutamic acid.

Glutamine is, for instance, substituted by D-citrulline and thio-L-citrulline. Glycine is, for instance, substituted by N-methylglycine, t-butylglycine, N-methylglycine, and D-allylglycine. Histidine is, for instance, substituted by 3-(3-methyl-4-nitrobenzyl)-L-histidine methyl ester. Isoleucine is, for instance, substituted by isodesmosine, N-methylisoleucine, and allo-isoleucine. Leucine is, for instance, substituted by norleucine, desmosine, and 5,5,5-trifluoro-leucine. Lysine is, for instance, substituted by 6-N-methyllysine, 2-aminoheptanoic acid, N-acetyl lysine, hydroxylysine, and allo-hydroxylysine. Methionine is, for instance, substituted by methionin sulfoxide. Phenylalanine is, for instance, substituted by p-amino-L-phenylalanine, 3-benzothienyl alanine p-bromophenylalanine, p-acyl-L-phenylalanine, 2-fluorophenylalanine, 3-fluorophenylalanine, and 4-fluorophenylalanine. Proline is, for instance, substituted by 3-hydroxyproline, 4-hydroxyproline, and 1-acetyl-4-hydroxy-L-proline. Serine is, for instance, substituted by homoserine, isoserine, 3-phenylserine. Threonine is, for instance, substituted by D-thyroxine and allo-threonine. Tryptophan is, for instance, substituted by 5-hydroxytryptophan, 5-methoxy-tryptophan, and 5-fluoro-tryptophan. Tyrosine is, for instance, substituted by O-methyl-L-tyrosine, O-4-allyl-L-tyrosine, and 3-chloro-tyrosine. Valine is, for instance, substituted by norvaline, N-methylvaline, and 3-fluoro-valine.

As used herein, a “polypeptide” refers to peptides, polypeptides and peptidomimetics that comprise multiple amino acids. The terms “polypeptide” and “peptide” are used interchangeably. The smallest polypeptides according to the disclosure of the present invention demonstrates to have a targeted preference for a particular bacteria, hence they interact with a specific quorum sensing motif of that particular bacteria, and have a length of 5 amino acids. However, the amino acid sequences or variants thereof are part of larger polypeptides, i.e., of polypeptides that have been N-terminally and/or C-terminally extended by additional amino acids. The amino acid sequences or variants of the disclosed polypeptides are N-terminally and/or C-terminally modified, preferably by comprising an N- and/or C-terminal elongating group. Alternatively, the amino acid sequences or variants thereof are N- and/or C-terminally extended. The polypeptides according to the disclosure, therefore, comprise at least 35 amino acids, and may comprise up to 1000 amino acids. However, smaller polypeptides are preferred in order to keep production costs as low as possible. Preferably, the polypeptides according to the disclosure is 35 to 200 amino acids in length, more preferably 35 to 100 amino acids, more preferably 35 to 50 amino acids.

As used herein, “peptidomimetic” refers to a compound containing non-peptidic structural elements, which compound mimics the antimicrobial/antibacterial, and/or anti-inflammatory properties of the disclosed polypeptides. Hence, these polypeptides may also comprise non-peptidic structural elements. Such non-peptidic structural elements may be present in all of the disclosed amino acid sequences, or in a variant thereof as defined herein, as a result of substitution of modification of one or more amino acids of the sequence or variant. Alternatively, the disclosed polypeptides of the present invention may comprise non-peptidic structural elements outside the amino acid sequences, or in a variant thereof as defined herein, i.e., in the optional N- and/or C-terminal elongating groups. A non-peptidic structural element in a peptidomimetic is typically a modification of one or more existing amino acids.

Preferred peptidomimetics are obtained by structural modification of polypeptides of the disclosure, for instance, using unnatural amino acids such as defined hereinabove, conformational restraints, cyclization of the polypeptide, isosteric replacement or other modifications. The amino acid sequence of a polypeptide according to the disclosure thus optionally comprises one or more modifications. Such polypeptide may be modified by natural processes, such as posttranslational processing, or by chemical modification techniques. Modifications may be inserted at any location in the polypeptide, including in the polypeptide backbone, amino acid side-chains and at the N- or C-terminus.

A single polypeptide may contain multiple types of modifications or several modification of a single type. Modifications include acetylation, amidation, acylation, phosphorylation, methylation, demethylation, ADP-ribosylation, disulfide bond formation, ubiquitination, gammacarboxylation, glycosylation, hydroxylation, iodination, oxidation, pegylation and sulfation. In addition, the polypeptides according to the disclosure may be provided with a label, such as biotin, fluorescein or flavin, a lipid or lipid derivative, a sugar group. The polypeptides according to the disclosure can further be provided with another targeting moiety other than the aforementioned quorum sensing motifs of a species-specific bacteria. Such other targeting moieties are preferably cell binding domains (CBDs) of bacteriophages.

As described herein above, the disclosed polypeptides of the present invention may be modified by chemical modification techniques known in the art. The modifications of the disclosed polypeptides can be introduced during or at the end of synthesis of the polypeptide. For instance, when the polypeptide is synthesized using solid-phase synthesis technique, N-terminal Palmitoylation can be performed at the end by reacting the amino acid sequence acid. As another example, C-terminal amidation, is, for instance, performed using a special kind of resin in solid-phase peptide synthesis, such as the commercially available MBHA, Rink or Sieber resins. These resins comprise a chemical handle from which amidated (poly)peptides are released during the cleavage. These and other methods of modifying polypeptides are known to any person skilled in the art.

It's an advantage of the present invention that salts of the disclosed polypeptides are also within the scope of the invention. Such salts include, but are not limited to, acid addition salts and base addition salts. As used herein, “pharmaceutically acceptable salt” of a polypeptide refers to a salt that retains the desired antimicrobial, antibacterial, and/or anti-inflammatory activity of the polypeptides, and is suitable for administration to humans or animals. Methods for the preparation of salts of polypeptides are known in the art and generally involve mixing of the polypeptide with a pharmaceutically acceptable acid or base, for instance, by reacting the free acid or free base forms of the product with one or more equivalents of the appropriate acid or base in a solvent or medium in which the salt is insoluble, or in a solvent such as water, which is then removed in vacuo or by freeze-drying, or by exchanging the cations of an existing salt for another cation on a suitable ion exchange resin. Examples of pharmaceutically acceptable acids and bases include organic and inorganic acids such as formic acid, acetic acid, propionic acid, lactic acid, glycolic acid, oxalic acid, pyruvic acid, succinic acid, maleic acid, malonic acid, trifluoroacetic acid, cinnamic acid, sulfuric acid, hydrochloric acid, hydrobromic acid, nitric acid, perchloric acid, phosphoric acid, and thiocyanic acid, which form ammonium salts with free amino groups of polypeptides, and bases that form carboxylate salts with free carboxylic groups of polypeptides, such as ethylamine, methylamine, dimethylamine, triethylamine, isopropylamine, diisopropylamine, and other mono-, di- and trialkylamines, and arylamines.

The polypeptides according to the disclosure can be prepared by various methods. For instance, they can be synthesized by commonly used solid-phase synthesis methods as originally described by Merrifield [32], or by Stewart [33], or as described in detail by Robert D. Waldup [34]. By using standard peptide synthesis methodology, it would be possible to substitute unnatural amino acids, such as Damino acids, for natural amino acids to enhance the stability or efficacy of the peptide in a manufactured product. Solid-phase synthesis methods are particularly suitable for synthesis of polypeptides or relatively short length, such as polypeptides with a length of up to about 70 amino acids in large-scale production and are well known in the art.

Alternatively, the polypeptides of the disclosure can be prepared using recombinant techniques well known in the art in which a nucleotide sequence encoding the polypeptide is expressed in host cells. Standard recombinant DNA and molecular cloning techniques used here are well known in the art and are described by Sambrook, J., Fritsch, E. F. and Maniatis, T. [35]; and by Silhavy, T. J., Bennan, M. L. and Enquist, L. W. [36]. Methods for recombinant synthesis of the peptides of the invention are also described in U.S. Patent Application No. 60/496,122, which is incorporated herein by reference.

Methods for recombinant synthesis of the polypeptides of the present invention include the preparation of synthetic genes by, for example, in vitro chemical synthesis of the genes using conventional methods as known in the art. “Synthetic genes” can be assembled from oligonucleotide building blocks that are chemically synthesized using procedures known to those skilled in the art. These building blocks are ligated and annealed to form gene segments that are then enzymatically assembled to construct the entire gene. The oligonucleotides necessary may be determined by backtranslating from the amino acid sequence of the peptide being synthesized.

“Chemically synthesized”, as related to a sequence of DNA, means that the component nucleotides were assembled in vitro. Manual chemical synthesis of DNA may be accomplished using well established procedures, or automated chemical synthesis can be performed using one of a number of commercially available machines. Various commercial synthetic apparatuses are available, such as the automated synthesizer from Applied Biosystems. Accordingly, the coding sequences can be tailored for optimal gene expression based on optimization of nucleotide sequence to reflect the “codon bias” of the host cell. The skilled artisan is well aware of the codon-bias exhibited by a specific host cell in usage of nucleotide codons to specify a given amino acid. Therefore, when synthesizing a gene for expression in a host cell, it is desirable to design the gene such that its frequency of codon usage approaches the frequency of preferred codon usage of the host cell. The skilled artisan appreciates the likelihood of successful gene expression if codon usage is biased towards those codons favored by the host. Determination of preferred codons can be based on a survey of genes derived from the host cell where sequence information is available. Accordingly, in the instant invention, if Escherichia coli (E. coli) were used as the expression host, codon bias for enteric bacteria could be utilized as the basis for synthesizing the nucleic acid sequences encoding the polypeptides such that optimal expression would be obtained in E. coli.

The synthetic genes may comprise, in addition to the peptide sequence, a fusion carrier peptide linked to the sequence encoding the antimicrobial peptide. The fusion carrier peptide may protect the host cell during expression from the toxic effects of the antimicrobial peptide. The fusion carrier peptide may also provide a signal sequence to direct export of an expressed antimicrobial peptide, or it may provide a means for subsequent purification of the expressed peptide.

The polypeptides of the present invention may also be synthesized as concatemers within a gene. The term “concatemer” herein refers to multiple copies of a given unit as tandem repeats. The multiple copies (multimers) may be separated by intervening sequences that provide, for example, cleavage sites for post-expression peptide recovery. For example, a gene might comprise multiple copies of the peptide described by Formula I:

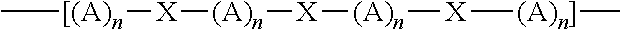

wherein X represents an intervening sequence between two or more copies of the (A)ₙ sequence for the antimicrobial peptide.

The polypeptides of the present invention may also be synthesized by solution-phase synthesis according to methods described by Paul Lloyd-Williams, Fernando Albericio, and Ernest Giralt and Miklos Bodanszky, Agnes Bodanszky [38]. For large-scale peptide synthesis, the shorter the length of the peptide sequence, the more amenable it is to large scale solution-phase synthesis [39].

In order to express the polypeptides in a suitable host cell, the DNA sequence encoding the peptide is operably linked to a suitable promoter. The term “operably linked” refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

“Promoter” refers to a DNA sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental or physiological conditions. Promoters that cause a gene to be expressed in most cell types at most times are commonly referred to as “constitutive promoters”. It is further recognized that since in most cases the exact boundaries of regulatory sequences have not been completely defined, DNA fragments of different lengths may have identical promoter activity.

The term “expression”, as used herein, refers to the transcription and stable accumulation of sense (mRNA) or antisense RNA derived from the nucleic acid fragment of the invention. Expression may also refer to translation of mRNA into a polypeptide.

The DNA sequence of the peptide may be operably linked to a promoter in a suitable vector, plasmid or cassette. The terms “plasmid”, “vector” and “cassette” refer to an extra chromosomal element often carrying genes which are not part of the central metabolism of the cell, and usually in the form of circular double-stranded DNA molecules. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

“Transformation cassette” refers to a specific vector containing a foreign gene and having elements in addition to the foreign gene that facilitate transformation of a particular host cell.

“Expression cassette” refers to a specific vector containing a foreign gene and having elements in addition to the foreign gene that allow for enhanced expression of that gene in a foreign host.

“Transformation” refers to the transfer of a nucleic acid fragment into the genome of a host organism, resulting in genetically stable inheritance. Host organisms containing the transformed nucleic acid fragments are referred to as “transgenic” or “recombinant” or “transformed” organisms.

The polypeptides of the present invention may be expressed from the plasmid in a suitable host, or the gene encoding the peptide may be incorporated into the host's chromosome. Host cells preferred for expression of the instant genes and nucleic acid molecules are microbial hosts that can be found broadly within the fungal or bacterial families and which grow over a wide range of temperature, pH values, and solvent tolerances. For example, it is contemplated that any bacteria, yeast, algae and filamentous fungi will be suitable hosts for expression of the present nucleic acid fragments. Because transcription, translation, and the protein biosynthetic apparatus is the same irrespective of the cellular feedstock, functional genes are expressed irrespective of carbon feedstock used to generate cellular biomass.

Large-scale microbial growth and functional gene expression may utilize a wide range of simple or complex carbohydrates, organic acids and alcohols, and saturated hydrocarbons such as methane or carbon dioxide in the case of photosynthetic or chemoautotrophic hosts. However, the functional genes may be regulated, repressed or depressed by specific growth conditions, which may include the form and amount of nitrogen, phosphorous, sulfur, oxygen, carbon or any trace micronutrient including small inorganic ions. In addition, the regulation of functional genes may be achieved by the presence or absence of specific regulatory molecules that are added to the culture and are not typically considered nutrient or energy sources. Growth rate may also be an important regulatory factor in gene expression.

Examples of suitable host strains include, but are not limited to: fungal or yeast species such as Aspergillus, Trichoderma, Saccharomyces, Pichia, Candida and Hansenula; or bacterial species such as Salmonella, Bacillus, Acinetobacter, Rhodococcus, Streptomyces, Escherichia, Pseudomonas, Methylomonas, Methylobacter, Alcaligenes, Synechocystis, Anabaena, Thiobacillus, Methanobacterium, Klebsiella, Burkholderia, Sphingomonas, Brevibacteium, Corynebacterium, Mycobacterium, Arthrobacter, Nocardia, Actinomyces, and Comamonas.

Microbial expression systems and expression vectors containing regulatory sequences that direct high level expression of foreign proteins are well known to those skilled in the art. Any of these could be used to construct chimeric genes for production any of the gene products of the instant sequences. These chimeric genes could then be introduced into appropriate microorganisms via transformation to provide high level expression of the enzymes.

Vectors or cassettes useful for the transformation of suitable host cells are well known in the art. Typically the vector or cassette contains sequences directing transcription and translation of the relevant gene, a selectable marker, and sequences allowing autonomous replication or chromosomal integration. Suitable vectors comprise a region 5′ of the gene which harbors transcriptional initiation controls and a region 3′ of the DNA fragment which controls transcriptional termination. It is most preferred when both control regions are derived from genes homologous to the transformed host cell, although it is to be understood that such control regions need not be derived from the genes native to the specific species chosen as a production host.

Initiation control regions or promoters, which are useful to drive expression of the instant ORF's in the desired host cell, are numerous and familiar to those skilled in the art. Virtually any promoter capable of driving these genes is suitable for the present invention including but not limited to: CYC1, HIS3, GAL1, GAL10, ADH1, PGK, PHO5, GAPDH, ADC1, TRP1, URA3, LEU2, ENO, TPI (useful for expression in
Saccharomyces); AOX1 (useful for expression in Pichia); and lac, ara, tet, trp, IPL, IPR, T7, tac, and trc (useful for expression in Escherichia coli) as well as the amy, apr, and npr promoters, and various phage promoters useful for expression in Bacillus.

Termination control regions may also be derived from various genes native to the preferred hosts. Optionally, a termination site may be unnecessary; however, it is most preferred if included.

The present invention can also be used to transform a suitable plant host with the gene(s) encoding the antimicrobial peptides or by means of transient expression which is well known by a person skilled in the art. Virtually any plant host that is capable of supporting the expression of an antimicrobial peptide gene will be suitable, however crop plants are preferred for their ease of harvesting and large biomass. Suitable plant hosts will include but are not limited to both monocots and dicots such as soybean, rapeseed (Brassica napus, B. campestris), sunflower (Helianthus annus), cotton (Gossypium hirsutum), corn, tobacco (Nicotiana Benthamiana), alfalfa (Medicago sativa), wheat (Triticum sp.), barley (Hordeum vulgare), oats (Avena sativa, L), sorghum (Sorghum bicolor), rice (Oryza sativa), Arabidopsis, cruciferous vegetables, melons, carrots, celery, parsley, tomatoes, potatoes, strawberries, peanuts, grapes, grass seed crops, sugar beet, sugar cane, canola, millet, beans, peas, rye, flax, hardwood trees, softwood trees, and forage grasses.

Where commercial production of the disclosed polypeptides of the present invention is desired, a variety of culture methodologies may also be applied. For example, large-scale production from a recombinant microbial host may be produced by both batch and continuous culture methodologies.

A classical batch culturing method is a closed system where the composition of the medium is set at the beginning of the culture and not subjected to artificial alterations during the culturing process. Thus, at the beginning of the culturing process the medium is inoculated with the desired organism or organisms and growth or metabolic activity is permitted to occur adding nothing to the system. Typically, however, a “batch” culture is batch with respect to the addition of carbon source and attempts are often made at controlling factors such as pH and oxygen concentration. In batch systems the metabolite and biomass compositions of the system change constantly up to the time the culture is terminated. Within batch cultures cells moderate through a static lag phase to a high growth log phase and finally to a stationary phase where growth rate is diminished or halted. If untreated, cells in the stationary phase will eventually die. Cells in log phase are often responsible for the bulk of production of end product or intermediate in some systems.

A variation on the standard batch system is the Fed-Batch system. Fed-Batch culture processes are also suitable in the present invention and comprise a typical batch system with the exception that the substrate is added in increments as the culture progresses. Fed-Batch systems are useful when catabolite repression is apt to inhibit the metabolism of the cells and where it is desirable to have limited amounts of substrate in the media. Measurement of the actual substrate concentration in FedBatch systems is difficult and is therefore estimated on the basis of the changes of measurable factors such as pH, dissolved oxygen and the partial pressure of waste gases such as CO2. Batch and Fed-Batch culturing methods are common and well known in the art and examples may be found in Thomas D. Brock in Biotechnology [40], herein incorporated by reference.

Fermentation media in the present invention must contain suitable carbon substrates. Suitable substrates may include but are not limited to monosaccharides such as glucose and fructose, oligosaccharides such as lactose or sucrose, polysaccharides such as starch or cellulose or mixtures thereof and unpurified mixtures from renewable feedstocks such as cheese whey permeate, cornsteep liquor, sugar beet molasses, and barley malt. The carbon substrates may also comprise, for example, alcohols, organic acids, proteins or hydrolyzed proteins, or amino acids. Additionally the carbon substrate may also be one-carbon substrates such as carbon dioxide or methane for which metabolic conversion into key biochemical intermediates has been demonstrated. In addition to one and two carbon substrates, methylotrophic organisms are also known to utilize a number of other carbon containing compounds such as methylamine and glucosamine, as well as methanol and a variety of amino acids for metabolic activity. For example, methylotrophic yeast are known to utilize the carbon from methylamine to form trehalose or glycerol [41].

Commercial production of the disclosed polypeptides in the present invention may also be accomplished with a continuous culture. Continuous cultures are open systems where a defined culture medium is added continuously to a bioreactor and an equal amount of conditioned medium is removed simultaneously for processing. Continuous cultures generally maintain the cells at a constant high liquid phase density where cells are primarily in log phase growth. Alternatively, continuous culture may be practiced with immobilized cells where carbon and nutrients are continuously added, and valuable products, by-products or waste products are continuously removed from the cell mass. Cell immobilization may be performed using a wide range of solid supports composed of natural and/or synthetic materials.

Continuous or semi-continuous culture allows for the modulation of one factor or any number of factors that affect cell growth or end product concentration. For example, one method will maintain a limiting nutrient such as the carbon source or nitrogen level at a fixed rate and allow all other parameters to moderate. In other systems a number of factors affecting growth can be altered continuously while the cell concentration, measured by medium turbidity, is kept constant. Continuous systems strive to maintain steady state growth conditions and thus the cell loss due to medium being drawn off must be balanced against the cell growth rate in the culture. Methods of modulating nutrients and growth factors for continuous culture processes as well as techniques for maximizing the rate of product formation are well known in the art of industrial microbiology and a variety of methods are detailed by Brock, supra.

As is well known to those skilled in the art, whole microbial cells can be used as catalyst without any pretreatment such as permeabilization. Alternatively, the whole cells may be permeabilized by methods familiar to those skilled in the art (e.g., treatment with toluene, detergents, or freeze thawing) to improve the rate of diffusion of materials into and out of the cells.

The polypeptides according to the disclosure of the present invention exhibit a number of activities that can be advantageously used in both therapeutic and nontherapeutic applications. In particular, polypeptides according to the disclosure are useful in counteracting various microbial infections, in particular bacterial infections. The unique effect of the polypeptides of the disclosure on (biofilm) infections is three-fold: they will 1) prevent biofilm formation and disperse existing biofilms, 2) kill the bacteria at and around the site of release, and 3) orchestrate immune responses by neutralizing proinflammatory microbial endotoxins such as LTA, and PG and activating macrophages to enhance their phagocytic and microbicidal activity. This immune control is necessary to prevent the tissue surrounding to become a novel niche for the pathogens. The polypeptides of the disclosure are active against a species-specific bacteria, including those that are resistant to conventional antibiotics. Provided, thus, are pharmaceutical compositions comprising a polypeptide according to the disclosure or a pharmaceutically acceptable salt thereof, and at least one pharmaceutically acceptable carrier, diluent and/or excipient. Also provided are pharmaceutical compositions comprising a nucleic acid molecule or vector according to the disclosure and at least one pharmaceutically acceptable carrier, diluent and/or excipient.

The disclosure further provides polypeptides according to the disclosure for use as a medicament. Further provided is a nucleic acid molecule comprising a nucleic acid sequence encoding a polypeptide according to the disclosure for use as a medicament. The medicament can be a therapeutic or a prophylactic agent.

The polypeptides and compositions thereof can also be used as preservatives or sterilants for articles susceptible to microbial contamination. The oligopeptides of the present invention and compositions thereof can be administered to a target cell or host by direct or indirect application. For example, the peptide may be administered topically, systemically or as a coating. The peptides of the present invention and compositions thereof may also be bound to or incorporated into substrates to provide antimicrobial substrates to reduce or inhibit microbial contamination of the substrate. The present invention also provides articles comprising the antimicrobial substrates of the invention.

Substrates suitable for the present invention include polymers selected from the group consisting of latex, polyvinyl chloride, polyimide, polyesters, polyethylene, polypropylene, polyamides, polyacrylates, polyolefins, polysaccharides, polyurethane, polysulfone, polyethersulfone, polycarbonate, fluoropolymers, cellulosics, synthetic rubber, silk, silicone, and mixtures or blends thereof. Additional polymer substrates are also functionalized polymer substrates comprising the aforementioned polymers and that additionally contain, or may be functionalized to contain, active groups with which peptides may react, and which allow for immobilization of the peptides. Examples of active groups include, but are not limited to: acrylic acid, acetal, hydroxyl, amines, epoxides, carboxylates, anhydrides, isocyanates, thioisocyanates, azides, aldehydes, halides, acyl halides, aryl halides and ketones at 1 to 50% by weight of the polymer. Various methods of protein or peptide immobilization are described in Protein Immobilization by Richard F. Taylor [42], herein incorporated by reference.

Substrates suitable for the present invention also include ceramics, glass, metal, metal oxides, and composites comprised of ceramics, glass, metal or metal oxides plus polymers as described above. Suitable metals include steel, stainless steel, aluminum, copper, titanium, alloys thereof, and combinations thereof.

The polypeptides of the present invention can also be impregnated, coated with, or covered, and used in or as medical materials, devices, or implants, such as bandages, adhesives, gauze strips, gauze pads, syringe holders, catheters such as peripheral IV catheters and central venus catheters comprised of either polyurethane or silicon, sutures, urinary catheter ostomy ports, orthopedic fixtures, orthopedic pins, pacemaker leads, defibrillator leads, ear canal shunts, vascular stents, cosmetic implants, ENT implants, staples, implantable pumps, hernia patches, plates, screws, blood bags, external blood pumps, fluid administration systems, heart-lung machines, dialysis equipment, artificial skin, artificial hearts, ventricular assist devices, hearing aids, vascular grafts, pacemaker components, hip implants, knee implants, and dental implants.

In the hygiene area, the polypeptides of the present invention can also be used in personal hygiene garments such as diapers, incontinence pads, sanitary napkins, sports pads, tampons and their applicators; and health care materials such as antimicrobial wipes, baby wipes, personal cleansing wipes, cosmetic wipes, diapers, medicated wipes or pads (for example, medicated wipes or pads that contain an antibiotic, a medication to treat acne, a medication to treat hemorrhoids, an anti-itch medication, an anti-inflammatory medication, or an antiseptic).

The polypeptides of the present invention can also be used in combination with biodegradable materials in the form of a coating. In particular, such biodegradable coating comprises a material selected from the group consisting of PLA (polylactic acid), PGA (polyglycolic acid), polycaprolactone (PCA), polyethylene oxide (PEO), polydioxanone (PDS), polycaprolactone (PCL), polypropylene fumarate, polymers derived from lactones, such as lactide, glycolide and caprolactone, carbonates such as trimethylene carbonate and tetramethylene carbonate, dioxanones, ethylene glycol, polyester amide (PEA) ethylene oxide, esteramides, γ-hydroxyvalerate, β-hydroxypropionate, α-hydroxy acid, hydroxybuterates, hydroxy alkanoates, polyimide carbonates, polyurethanes, polyanhydrides, and combinations thereof, polysaccharides such as hyaluronic acid, chitosan and cellulose, and proteins such as gelatin and collagen.

As used herein, the polypeptides of the present invention can be used in subjects. A “subject” is a human or an animal. Subjects include, but are not limited to, mammals such as humans, pigs, ferrets, seals, rabbits, cats, dogs, cows and horses, and birds such as chickens, ducks, geese and turkeys. In a preferred embodiment of the disclosure, a subject is a mammal. In a particularly preferred embodiment, the subject is a human.

The disclosure also provides a method for inhibiting the growth of a microbe, e.g., a bacterium with a polypeptide or pharmaceutical composition according to the disclosure. The contacting can be performed in vivo and in vitro.

The polypeptides and pharmaceutical compositions according to the disclosure are effective in treating a variety of microbial infections, such as various viral, bacterial and fungal infections. For example, the polypeptides and pharmaceutical compositions are effective in treating species-specific pathogenic bacteria. Examples of pathogenic bacteria that may cause infections in humans or animals that are treatable with polypeptides and compositions of the disclosure include, Cutibacterium acnes, Staphylococcus Aureus, Methicillin-Resistant Staphylococcus aureus (MRSA), Gardnerella Vaginalis, but the scope of this invention is not limited to other bacterial pathogens that may cause infections in humans or animals such as Listeria, Escherichia, Chlamydia, Rickettsial bacteria, Mycobacteria, Staphylococci, Streptococci, Pneumonococci, Meningococci, Klebsiella, Pseudomonas, Legionella, Diphtheria, Salmonella, Bacilli, Vibrio cholerae, Tetanus, Clostridium, Bacillus, Yersinia, and Leptospira bacteria.

The compositions containing the polypeptides of the present invention can be administered for prophylactic and/or therapeutic treatments. In therapeutic applications, polypeptides or compositions are administered to a subject, preferably a human, already suffering from a disease in an amount sufficient to counteract the symptoms of the infection or the condition resulting from the infection and its complications. In prophylactic applications, polypeptides or compositions are administered to a subject, for instance, a human or animal at risk of suffering from a bacterial infection in an amount sufficient to prevent infection or at least inhibit the development of an infection. The polypeptide is typically present in a pharmaceutical composition according to the disclosure in a therapeutic amount, which is an amount sufficient to remedy a condition or disease, particularly symptoms associated with a microbial or parasitic infection. Typical doses of administration of a polypeptide according to the disclosure or combinations of at least two thereof are between 0.001 and 10 mg polypeptide per kg body weight, depending on the size of the polypeptide of the present invention. More specifically, dosage of a polypeptide in the topical formula disclosed in the present invention may comprise from about 0.0001% to about 1% by weight of the composition, preferably 0.001 to 1% by weight of the composition, more preferably 0.01 to 1% by weight of the composition.

The disclosed polypeptides and pharmaceutical composition of the present invention are particularly suitable for topical application, e.g., in the treatment or prevention of skin infections, wound infections and urinary tract infections. As detailed herein before, the polypeptides of the disclosure are capable of preventing biofilm formation and disperse existing biofilms, thereby killing specific bacteria at and around the site of biofilm formation, and modulate immune responses by neutralizing proinflammatory microbial endotoxins. Further provided is the use of the polypeptides, pharmaceutical composition and/or nucleic acid molecules of the present invention in the manufacture of a pharmaceutical composition for the treatment or prevention of skin infection. The disclosure further provides a method for the treatment of a subject suffering from skin infection, wound infection and/or urinary tract infection comprising administering to the subject a therapeutically effective amount of the polypeptides according to the disclosure, a pharmaceutical composition according to the disclosure, or nucleic acid molecules according to the disclosure.

The disclosed polypeptides of the present invention are advantageously incorporated in a controlled release and/or targeted delivery carrier.

As used herein, the term “controlled release” refers to the release of the polypeptide of the disclosure in a time-dependent manner. In one embodiment, controlled release refers to slow release.

As used herein, the term “targeted delivery” refers to the release of the polypeptide of the disclosure in a site-directed manner. Use of a controlled release vehicle has the advantage that frequent administration such as by injection of the polypeptide of the disclosure can be avoided. Use of a targeted delivery vehicle has the advantage that the polypeptide of the disclosure is effectively delivered to and/or retained at a site of interest in a subject's body, such as a site of inflammation or a site of infection. Preferably, a polypeptide of the disclosure is targeted to a site infected by bacteria.

Controlled release and/or targeted delivery carriers are well known in the art. Non limiting examples of controlled release and/or targeted delivery vehicles are nanoparticles, microparticles, nanocapsules, microcapsules, liposomes, microspheres, hydrogels, polymers, lipid complexes, serum albumin, antibodies, cyclodextrins and dextrans.

Controlled release is, for instance, provided by incorporating a polypeptide of the disclosure in or on the surface of such carrier. The carriers are of materials that form particles that capture a polypeptide of the disclosure and slowly degrade or dissolve in a suitable environment, such as aqueous, acidic or basic environment or body fluids, and thereby release the polypeptide.

Targeted delivery is, for instance, achieved by providing a carrier with targeting groups on the surface thereof Examples of such carrier comprising targeting groups are antibody-functionalized carriers, carriers having a site-specific ligand and carriers having a positive or negative surface charge. Preferred particles for controlled release and/or targeted delivery are nanoparticles, i.e., particles in the range of about 1 to 500 nm in diameter, preferably up to about 300 nm in diameter, and liposomes, optionally provided with targeting groups.

Preferred targeted delivery and/or controlled release carriers for delivering the polypeptide of the present invention, are of biodegradable material. “Biodegradable” as used herein refers to molecules that degrade under physiological conditions. This includes molecules that are hydrolytically degradable and molecules that require enzymatic degradation. Suitable biodegradable materials include, but are not limited to, biodegradable polymers and natural biodegradable material such as PLA (polylactic acid), PGA (polyglycolic acid), polycaprolactone (PCA), polyethylene oxide (PEO), polydioxanone (PDS), polycaprolactone (PCL), polypropylene fumarate, polymers derived from lactones, such as lactide, glycolide and caprolactone, carbonates such as trimethylene carbonate and tetramethylene carbonate, dioxanones, ethylene glycol, polyester amide (PEA) ethylene oxide, esteramides, γ-hydroxyvalerate, β-hydroxypropionate, α-hydroxy acid, hydroxybuterates, hydroxy alkanoates, polyimide carbonates, polyurethanes, polyanhydrides, and combinations thereof, polysaccharides such as hyaluronic acid, chitosan and cellulose, and proteins such as gelatin and collagen.

Although the polypeptides of the present invention are potent antimicrobial agents for use as is, they can be combined with known antimicrobial agents, such as conventional anti-infectives, such as antibiotics, or other antimicrobial peptides, and antibodies and chemicals, e.g., sensitizers and nanoparticles. Such combination may result in an increased antimicrobial activity or broaden the spectrum of activity. The polypeptides of the present invention may, for instance, be combined with penicillins, cephalosporins, macrolides, fluoroquinolones, sulfonamides, tetracyclines and/or aminoglycosides for treating bacterial infections.

Pharmaceutical compositions according to the disclosure comprise at least one pharmaceutically acceptable carrier, diluent or excipient. In a preferred embodiment, the suitable carrier is a solution, for example, saline. Examples of excipients that can be incorporated in tablets, capsules and the like are the following: a binder such as gum tragacanth, acacia, corn starch or gelatin; an excipient such as microcrystalline cellulose; a disintegrating agent such as corn starch, pregelatinized starch, alginic acid and the like; a lubricant such as magnesium stearate; a sweetening agent such as sucrose, lactose or saccharin; and a flavoring agent such as peppermint (e.g menthol). When the dosage unit form is a capsule, it may contain, in addition to materials of the above type, a liquid carrier such as fatty oil. Various other materials may be present as coatings or to otherwise modify the physical form of the dosage unit. For instance, tablets may be coated with shellac, sugar or both. A syrup or elixir may contain the active compound, sucrose as a sweetening agent, methyl and propyl parabens as preservatives, a dye and a flavoring such as cherry or orange flavor. A pharmaceutical composition according to the disclosure is preferably suitable for human use.

The pharmaceutical compositions described herein can be administered in a variety of different ways. Examples include administering a pharmaceutical composition comprising a polypeptide according to the disclosure and containing a pharmaceutically acceptable carrier via oral, intranasal, rectal, topical, intraperitoneal, intravenous, intramuscular, subcutaneous, subdermal, transdermal, intrathecal, and intracranial methods. For oral administration, the active ingredient can be administered in solid dosage forms, such as capsules, tablets, and powders, or in liquid dosage forms, such as elixirs, syrups, and suspensions.

Sterile compositions for injection can be formulated according to conventional pharmaceutical practice by dissolving or suspending the polypeptide of the disclosure in a vehicle for injection, such as water or a naturally occurring vegetable oil like sesame oil, coconut oil, peanut oil, cottonseed oil, etc., or a synthetic fatty vehicle like ethyl oleate or the like. Buffers, preservatives, antioxidants and the like may also be incorporated.

In a preferred embodiment, a pharmaceutical composition according to the disclosure is formulated for topical administration. “Topical administration” as used herein refers to application to a body surface such as the skin or mucous membranes to locally treat conditions resulting from microbial or parasitic infections. Examples of formulation suitable for topical administration include, but are not limited to, a cream, gel, ointment, lotion, foam, suspension, spray, aerosol, or powder aerosol. Topical medicaments can be epicutaneous, meaning that they are applied directly to the skin. Topical medicaments can also be inhalational, for instance, for application to the mucosal epithelium of the respiratory tract, or applied to the surface of tissues other than the skin, such as eye drops applied to the conjunctiva, or ear drops placed in the ear. The pharmaceutical composition formulated for topical administration preferably comprises at least one pharmaceutical excipient suitable for topical application, such as an emulgent, a diluent, a humectant, a preservative, a pH adjuster and/or water.

The disclosure will be explained in more detail in the following, non-limiting examples.

### DETAILED DESCRIPTION

### Examples

### Material and Methods

Synthesis of Fusion Peptides Targeting C. acnes (Cutibacterium acnes)

The peptides were synthesized in stepwise manner on a Biotage Alstra Plus synthesizer (Biotage) using a fluorenylmethyloxycarbonyl (FMOC) solid-phase peptide synthesis (SPPS) strategy with an H-Rink Amide ChemMatrix resin (0.45 mmol/g) (PCAS Biomatrix) as a solid support to obtain C-terminally amidated peptides. Introduction of N-terminal fragments of peptides was performed with palmitoyl chloride in the presence of a base method. Furthermore, the resin was swollen in N,N-dimethylformamide (DMF) for 20 min at 70° C. with an oscillating mixer.

At each coupling step, fmoc protected D- or L-amino acids were used and dissolved in DMF, and ethyl 2-cyano-2(hydroxyimino)acetate (OxymaPure) and carbodiiamide (DIC) were used as coupling reagents for 5 min at 75° C. The fmoc group was removed by treatment with piperidine (20% v/v) in DMF (first reaction at 45° C. for 2 min, then the second reaction at room temperature for 12 min).

The final cleavage was performed using a standard protocol (95% trifluoroacetic acid [TFA], 2.5% triisopropylsilane [TIS], and 2.5% H₂O) for 4 hrs at room temperature (22° C.). Peptides were precipitated in cold diethyl ether and purified by reverse-phase high-performance liquid chromatography (RP-HPLC) using a BioBasic C-8 column (Thermo Scientific) and a 20%-80% acetonitrile (in water with addition of 0.1% TFA to both solvents) gradient. The purified peptides were lyophilized, and the molecular masses of the peptides were analyzed by ultra-performance liquid chromatography-mass spectrometer (UHPLC-MS; Agilent 1260 Infinity, Agilent Technologies).

The following C. acnes targeting constructs were designed by SSPS:

(SEQ ID NO: 1/Wildtype BLP-7)
GIGGALLSAGKSALKGLAKGLAEHFAN
(SEQ ID NO: 2/BMBN-1)
(N-PALM)-GIGGALLSAGKSALKGLAKGLAEHFAN-(CONH2)
(SEQ ID NO: 3/BMBN-2)
(N-PALM)-ERNNF-GGG-GIGGALLSAGKSALKGLAKGLAEHFAN-
(CONH2)
D-form
(SEQ ID NO: 4/BMBN-3)
(N-PALM)-ERNNF-GGG-GIGGALLSAGKSALKGLAKGLAEHFAN-
(CONH2)
(SEQ ID NO: 5/BMBN-4)
(N-PALM)-EFGGQ-GGG-GIGGALLSAGKSALKGLAKGLAEHFAN-
(CONH2)
(SEQ ID NO: 6/BMBN-5)
(N-PALM)-ERNNF-GGG-GIGGALLSAGESALKGLAKGLAEHFAN-
(CONH2)
RI
(SEQ ID NO: 7/BMBN-6)
(N-PALM)-ERNNF-GGG-NAFHEALGKALGKLASEGASLLAGGIG-
(CONH2)
D-form
(SEQ ID NO: 8/BMBN-7)
(N-PALM)-ERNNF-GGG-GIGGALLSAGESALKGLAKGLAEHFAN-
(CONH2)

Of which:
(N-PALM) stands for N-terminal palmitoylation of the N-terminal
(CONH₂) stands for C-terminal amidation
RI stands for retro-inverso sequence
D-form stands for D-amino acids, the chiral form of L-amino acids
Letters in bold stand for amino acid substitution
GGG stands for a glycine rich short linker

The following clinical isolates of different types of bacteria were stored at −80° C. until used:

Cutibacterium acnes HL045PA1
Cutibacterium acnes RT6 HL110PA3
Cutibacterium acnes DSM 16379/KPA171202
Cutibacterium acnes HL096PA3
Cutibacterium granulosum DSM 20700
Cutibacterium avidum ATCC 25577
Staphylococcus aureus NCTC 8325/PS 47
Staphylococcus epidermis ATCC 35984/RP62A
Corynebacterium striatum 3012STDY7069329

Inoculi of mid-log phase bacteria were prepared by incubating the above isolated colonies from blood agar plates in Tryptic Soy Broth (TSB) medium (Becton Dickinson, Le Pont de Clax, France) for 2.5 hours and then diluted to the concentration needed.

### In Vitro Killing Assay:

For the in vitro killing assay on mid-log phase bacteria, each of the above strains were resuspended to a concentration of 1×10⁶ bacteria/ml in phosphate buffered saline (PBS). Subsequently, 200 μl was added to a concentration range of peptides Wildtype BLP-7, BMBN-1, BMBN-2, BMBN-3, BMBN-4, BMBN-5, BMBN-6, and BMBN-7 that were lyophilized in advance. Subsequently, the bacteria-peptide mixtures were incubated for 1 hour at 37° C. To establish the killing capacity and specificity of these peptides, the suspensions were serially diluted and plated onto DST agar plates to measure viable CFU counts. IC90, IC99 and IC99.9 values were calculated by linear regression analysis. Depicted in (FIGS. 2A and 2B) is the concentration of the peptide BMBN-7 that resulted in killing of 99.9% of C. acnes bacteria (1000 CFU/ml remaining) and showing BMBN-7 had no effect on the other bacteria tested, thus showing the effective specificity and binding ability towards C. acnes. The other constructs BMBN-2, BMBN-3, BMBN-5, BMBN-6 showed comparable results, with their specificity towards C. acnes only negligibly decreased. The BMBN-4 construct that contained a random sequence (EFGGQ) instead of the quorum sensing motif (ERNNF) resulted in total loss of specificity and remained to be broad spectrum towards killing the other bacterial species, as was the same with the wildtype BLP-7 and BMBN-1 constructs. (FIG. 2C) also shows that BMB-7 clearly outperforms the wildtype BLP-7 peptide as a result of its modifications. Results were pooled from 4 independent experiments concerning the above C. acnes strains (FIG. 2B) and 5 independent experiments concerning the other non C. acnes related strains (FIG. 2B continued). The results are expressed as mean±standard deviation (SD).

### Broth Microdilution Assay:

Procedures in line with the CLSI guidelines were performed to determine the MIC of peptides; wildtype BLP-7, BMBN-1, BMBN-2, BMBN-3, BMBN-4, BMBN-5, BMBN-6, BMBN-7 against C. acnes HL045PA1, RT6 HL110PA3, DSM 16379/KPA171202, and HL096PA3 isolates which consisted of 20 isolates each for each peptide respectively. Strains were grown for 18-24 hrs at 37° C. under 5% CO2. Direct suspension of the colonies were made in CAMHB and adjusted to OD625 0.08-0.1 which corresponds to 1˜2×10⁸ CFU/ml followed by serial ten-fold dilutions to give 1×10⁶ CFU/ml. 50 μL of bacterial suspension was then aliquoted to 96-well round bottom microtiter plates containing equal volume of serially diluted peptides to give final concentrations of peptides encompassing the range of 1.96-100 μg/ml. The MIC value for possible inactive peptides producing no inhibition in the range tested was denoted as >100 μg/ml. The plates were incubated for 18-24 hrs at 37° C. under 5% CO2. MIC was read as the concentration of peptide producing complete inhibition on the visible growth of the test organism. Results were pooled from three independent experiments. MIC range was defined as the range of concentrations where the peptides produce detectable antimicrobial activity. Effective percentage (EP) was the proportion of C. acnes isolates inhibited within the MIC range. The results were in line with previous published findings for the activity of wildtype BLP-7 against C. acnes (MIC=5 μg/ml), though all of the novel polypeptides managed to show an improved antibacterial propensity versus the wildtype BLP-7 (FIG. 3).

### Transmission Electron Microscopy (TEM):

An overnight culture of C. acnes HL045PA1 on porcine blood agar was passaged twice and directly suspended in Cation-Adjusted Mueller-Hinton Broth (CAMHB). The bacteria were prepared to 5×10¹⁰ CFU/ml as samples for TEM required high cell density of about 10⁸ to 10¹⁰ to be viewable, and treated with supra-concentration of peptides at 5 mg/ml for 4 hours at 37° C. under 5% CO2. Cells treated with only purified water was served as the untreated control. The cells were washed twice with CAMHB before overnight fixation in 5% (v/v) glutaraldehyde, two times postfix washes with cacodylate buffer, incubate, 2 hours incubation with osmium tetroxide buffer (OsO4 1:1 cacodylate) and washed twice with cacodylate buffer before overnight incubation in the same buffer.

Next, the samples were washed twice with double distilled water, 10 min of uranyl acetate incubation and washed twice with double distilled water before subjected to dehydration by gradual ethanol series: 30% for 10 min, 45% for 10 min, 60% for 10 min, 85% for 15 min and three rounds of absolute (100%) ethanol for 15 min.

Following this, samples were incubated with two rounds of propylene oxide for 15 min, propylene oxide 1:1 EPON for 1 hour, propylene oxide 1:3 EPON for 2 hours, overnight incubation with EPON, embedded in Agar 100 resin at 37° C. for 5 hours and maintained in 60° C. until viewing. Ultrathin sectioning were prepared on an Artos 3D Ultramicrotome (Leica microsystems), copper grids 3.05 mm (300 square mesh) (Agar Scientific) and stained with ethanol-based uranyl acetate and lead citrate for 5 min.

The prepared samples were viewed with an H-9500 (Hitachi) under standard operating conditions. A) Untreated control showing the normal shape of C. acnes while severe cell morphological changes following treatment with BMBN-7-treated C. acnes HL045PA1 cells were evident: (arrow a) cell wall/membrane breakages, (arrow al) loss of cell wall, (arrow b) large cavity between cell wall and plasma membrane, (arrow c) leakage of intracellular contents, (arrow d) breakdown of cytoplasm into inclusion bodies, and (arrow e) loss of cytoplasm. Bar indicates 100 nm. (FIG. 4)

### Hemolytic Activity:

A hemolytic assay was performed as previously described by Zelezetsky et al. [43]. Freshly drawn human erythrocytes were rinsed three times with phosphate buffered saline (PBS) and resuspended in PBS to 4% (v/v). One hundred microliters of the suspension was added to 96-well microtiter plate containing equal volume of peptides to give final concentrations of peptides encompassing the range of 2.00-250 μg/ml. PBS and 0.1% (v/v) Triton-X 100 were used as 0% and 100% hemolytic control respectively. Plates were incubated at 37° C. for 1 hour.

Subsequently, the plate was centrifuged and the supernatant was transferred to a new plate. The release of hemoglobin in the supernatant was monitored at absorbance of 450 nm using an Infinite M1000 Microplate reader (Tecan). Results were pooled from three independent experiments and expressed as mean±SD. HC₁₀ and HC₅₀ were defined as the peptide concentrations causing 10% and 50% hemolysis on human erythrocytes, respectively. Hmax was the percentage hemolysis observed at the maximum concentration as defined throughout this study (all peptides 250 μg/ml). BMBN-1, BMBN-2, BMBN-3, BMBN-4, BMBN5, BMBN-6 and BMBN7, all displayed no hemolytic activity at the range of concentrations tested (HC10>250 μg/ml, Hmax<1.0%) compared to the wildtype BLP7 peptide, though also within the range of negligible hemolytic activity, which can be attributed by their modifications (FIG. 5).

### Cytotoxicity Against Human Cell Lines:

An NL20 human lung bronchial normal epithelial cell line was used for cell cytotoxicity tests. NL20 cell line was grown in Ham's F12 medium (GIBCO) and media was supplemented with fetal bovine serum to 10% (v/v) as growth medium or 2.5% (v/v) as maintenance medium in cell cytotoxicity testing. The assay was performed as previously described by Lee et al [44]. NL20 cells were seeded overnight at 3×10⁴ cells/well in 96-well cell culture-treated flat bottom microtiter plate (Thermo Scientific) and treated with serial dilutions of polypeptides at final concentrations of 1.95-250 μg/ml. The polypeptides were tested from 0.05-10 μg/ml. Cell viability was detected by using CyQUANT™ LDH Cytotoxicity Assay (Invitrogen) and the colorimetric changes were read with an Infinite M1000 Microplate reader (Tecan) at OD 490 nm. Results were pooled from 8 independent experiments and expressed as mean±SD. IC50 was defined as the polypeptide concentration which resulted in 50% cell viability. Imax was the percentage of cell viable treated with the peptides at the maximum concentration as defined throughout this study (all polypeptides 250 μg/ml). (FIG. 6)

### Inhibition of Inflammatory Cytokines:

Inflammation during the pathogenesis of acne is accompanied by cascade events of inflammatory cytokine production, such as Tumor Necrosis Factor Alpha (TNF-α), and interleukins IL-8, and IL-12 [45, 46]. To evaluate the effects of C. acnes infection on the expression of inflammatory cytokines, the levels of TNF-α, IL-8, and IL-12 were assessed via RT-PCR and ELISA at different time points after the infection of RAW264.7 cells with C. acnes (strain HL045PA1). The mRNA expression level of both IL-8 and IL-12 was notably increased after 1 and 2 hours of infection and was further dramatically enhanced after 6 hours of infection. Moreover, the mRNA expression level of TNF-α was elevated significantly from 1 to 6 hours after infection, which was in stark contrast to that observed in the negative control group. In the ELISA assays, IL-8 was significantly upregulated at the early stage (2 hours) after infection, and the peak IL-8 concentration (at 24 hours) was notably greater than that observed in the control. In addition, the concentrations of IL-12 and TNF-α were not significantly different from those observed in the negative control group, reaching maximum values at 12 hours, after which they decreased. These results showed that the expression of inflammatory cytokines in RAW264.7 cells could be significantly upregulated via C. acnes infection.

To investigate the anti-inflammatory effects of the peptides, the expression of IL-8, IL-12, and TNF-α was determined via qRT-PCR and ELISA after the treatment of RAW264.7 cells with the peptides. The RAW264.7 cells were pretreated with the peptides wildtype BLP-7, and BMBN-7 for 1 hour and infected with C. acnes (strain HL045PA1) at a multiplicity of infection (MOI) of 10 or were treated with peptidoglycan (PG) as the positive control. The results indicated that the mRNA expression of IL-8, IL12, and TNF-α induced by C. acnes (strain HL045PA1) infection was significantly inhibited after the treatment with each of the peptides. At the same time, the mRNA expression of IL-8 and TNF-α caused by PG was also greatly reduced by the peptides, while the effects of the peptides on the expression of IL-12 caused by the PG treatment was not significantly altered. Moreover, the peptides could also significantly reduce the secretion of IL-8, IL-12, and TNF-α produced via the induction of macrophages by C. acnes (strain HL045PA1) and PG. Moreover, BMB-7 showed a higher decrease against the wildtype BLP-7 peptide. The results suggested that the peptides could notably inhibit C. acnes-induced inflammatory cytokine expression in RAW264.7 cells. (FIG. 7, graphs A-F, and FIG. 8, graphs A-F)

### Quantitative Biofilm Formation Assay

A quantitative biofilm formation assay was performed according to the crystal violet staining method as previously described by George A. O'Toole [47]. The 96-well tissue culture plate method was utilized for a quantitative evaluation of biofilm formation by 100 strains of antibiotic-resistant C. acnes. C. acnes strain HL045PA1 was used as the positive control, and tryptic soy broth (TSB) medium (Becton Dickinson) without bacteria was used as the negative control. Absorbance at 570 nm (OD570) was measured for each well to obtain quantitative data on biofilm formation as previously described by

Badmasti et al. [48]. The mean±standard deviation of the OD value of the negative control was defined as ODc. Based on the OD value, the strains were divided into the following four groups: the OD value of the test strain was compared with ODc, and OD570≤ ODc was negative for biofilm formation (−); ODc<OD570≤2×ODc indicates weak biofilm formation (+); 2×ODc<OD570≤4×ODc indicates moderate biofilm formation (++); 4×ODc<OD570 indicates strong biofilm formation (+++). Cutibacterium granulosum DSM 20700 was used as a positive control.

### Detection of the Minimum Biofilm Inhibition Concentration (MBIC) and Minimum Biofilm Eradication Concentration (MBEC)

In order to detect the inhibitory effect of polypeptides; wildtype BLP-7, and BMBN-7 on the growth of biofilms, a previously described method by Abouelhassan et al. was used with modifications [49]. Briefly, 250 μl of bacterial cells (1×10⁶ CFU/ml) of 10 strains (C. acnes 3, 4, 23, 25, 55, 61, 62, 63, 68, 78) (FIG. 9) with the strongest biofilm formation ability were inoculated in a Greiner polyethylene 96well plate (Sigma Aldrich); TSB culture medium containing no bacteria was used as the blank control, and the plate was incubated at 37° C. for 24 hours. The culture medium was subsequently removed, and wells were carefully washed with PBS three times to remove C. acnes bacteria.

Subsequently, 250 μl of TSB culture medium containing the aforementioned polypeptides in serial doubling dilutions was added to each well. TSB medium without any of the polypeptides was used as a negative control, and plates were incubated at 37° C. for 24 hours. If OD600<0.1, there was no bacterial growth, and the lowest concentration without bacterial growth at this time point was recorded; this is the Minimum Biofilm Inhibitory Concentration (MBIC). Then, the cells and polypeptides in the 96-well cell culture plate were washed with PBS, and 250 μl of TSB culture medium was added to each well.

The plates were incubated at 37° C. for 24 hours to re-grow the surviving biofilm bacteria. An OD600<0.1 indicated that there was no bacterial growth. The lowest concentration at which no bacterial growth was recorded is the minimal biofilm eradication concentration (MBEC).

In order to evaluate the eradication efficiency of the polypeptides on the C. acnes biofilm, the culture in the wells was removed and washed with PBS to remove non-adherent cells. The biofilm was quantified by the aforementioned method, and calculated using the equation:

$$( 1 - \frac{OD 570 of the test}{OD 570 of non - treated control} ) \times 100$$

As shown in (FIG. 10) the OD570 value of the positive control C. acnes strain HL045PA1 in the experimental plate was 1.283+0.019, and the negative control was 0.204±0.003. The OD570 value of the 100 resistant bacteria was between 0.1 and 1.9. All 100 strains of C. acnes were able to form biofilms. Among them, 27 strains (24.5%) had strong biofilm formation ability, 60 strains (60%) were moderate and 13 strains (13.5%) were weak.

Inhibition and Eradication of C. acnes Biofilm

As shown in (FIG. 10), the polypeptides; wildtype BLP-7, and BMBN-7 can inhibit the biofilm of the strains with the strongest C. acnes biofilm formation ability at 56-117 μg/ml (BMBN-7) and 38-109 μg/ml (wildtype BLP-7) respectively, and can eradicate the biofilm at 235-496 μg/ml (BMBN-1) and 224-485 μg/ml (wildtype BLP-7) respectively. In order to determine the effect of the polypeptides on the removal of biofilms, after the formation of mature biofilms, different concentrations of the polypeptides were added to the culture for 24 hours. The crystal violet staining method was used to measure the absorbance at 570 nm to calculate the clearance rate of different concentrations of the peptide on mature biofilms. The results showed that 1×MIC (4 μg/ml) could clear more than 20% of mature biofilms, with MBEC₅₀ of 14 μg/ml and MBEC₈₀ of 125 μg/ml (FIG. 11).

Topical Formulation for the Treatment of a Skin Infection Caused by C. acnes.

| | | |
|---|---|---|
| INGREDIENT NAME | FUNCTION | % W/W |

Phase A

| | | |
|---|---|---|
| Aqua (water) | Diluent | 72.30 |
| Pentylene Glycol | Humectant, Solvent | 1.50 |
| Glycerin | Humectant, Solvent | 0.75 |
| Sodium Hyaluronate | Humectant | 0.50 |
| Niacinamide | Active | 3.00 |
| Panthenol | Skin conditioning | 1.00 |
| Allantoin | Skin conditioning | 0.50 |
| Pentylene Glycol (and) | Functional | 0.75 |
| Methyl Diisopropyl Propionamide | | |
| Methyl Methacrylate Crosspolymer | Texturizing Agent | 0.50 |

Phase B

| | | |
|---|---|---|
| Polyacrylate-13 (and) | Thickener | 2.00 |
| Polyisobutene (and) | | |
| Polysorbate 20 | | |
| Hydroxyethyl Acrylate/ | Thickener | 1.50 |
| Sodium Acryloyldimethyl | | |
| Taurate Copolymer (and) | | |
| Polyisobutene (and) | | |
| PEG-7 Trimethylolpropane | | |
| Coconut Ether | | |
| C13-15 Alkane | Emollient | 10.00 |
| Eucalyptus Globulus Leaf Oil | Functional | 0.50 |
| Rosmarinus Officinalis (Rosemary) | Functional | 0.25 |
| Leaf Oil | | |
| Methylglucose Isostearate | Co-emulsifier | 0.15 |
| Bisabolol | Skin conditioning | 0.50 |
| Retinal* | Active | 1.00 |
| Phospholipids (and) BMBN-7* | Active | 2.00 |

Phase C

| | | |
|---|---|---|
| Phenoxyethanol, Ethylhexylglycerin | Preservative, | 1.00 |
| | Antimicrobial | |
| | stabilizer | |
| Ethylhexylglycerin | Antimicrobial | 0.30 |
| | booster | |

Phase D

| | | |
|---|---|---|
| Sodium Hydroxide (or) | Ph Adjuster | q.s. to pH |
| Triethanolamine | | 5.00-6.00 |

*Of which BMBN-7 content is 0.1% W/W

### BRIEF DESCRIPTION OF THE DRAWINGS

FIG. 1: Sequences of Wildtype BLP-7 and modifications thereof for the targeting of C. acnes.

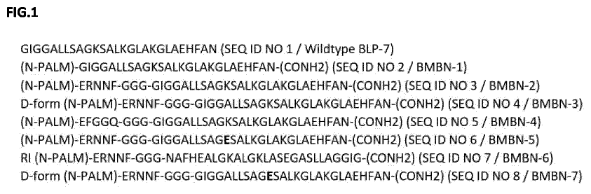

FIG. 2A: Killing curves of peptides wildtype BLP-7, BMBN-1, BMBN-2, BMBN-3, BMBN-4, BMBN-5, BMBN-6, BMBN-7 on C. acnes (strain HL045PA1) represented by the amount of CFU (colony-forming units) of C. acnes per ml of culture medium by different concentrations of the peptides.

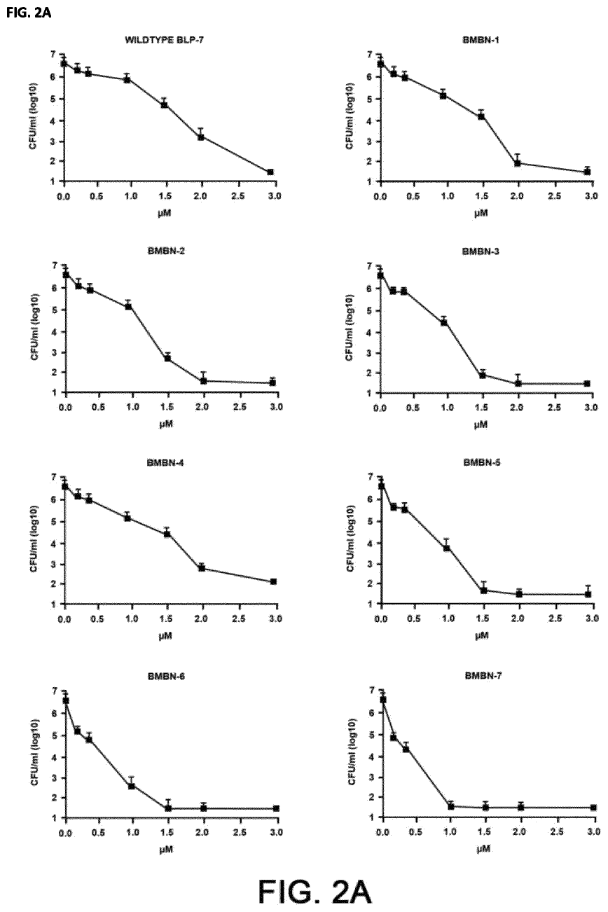

FIG. 2B: IC90, IC99 and IC99.9 (90%, 99% and 99.9% Inhibitory Concentration) values for the peptides tested on C. acnes, C. granulosum, C. avidum, S. aureus, S. epidermis, and C. striatum.

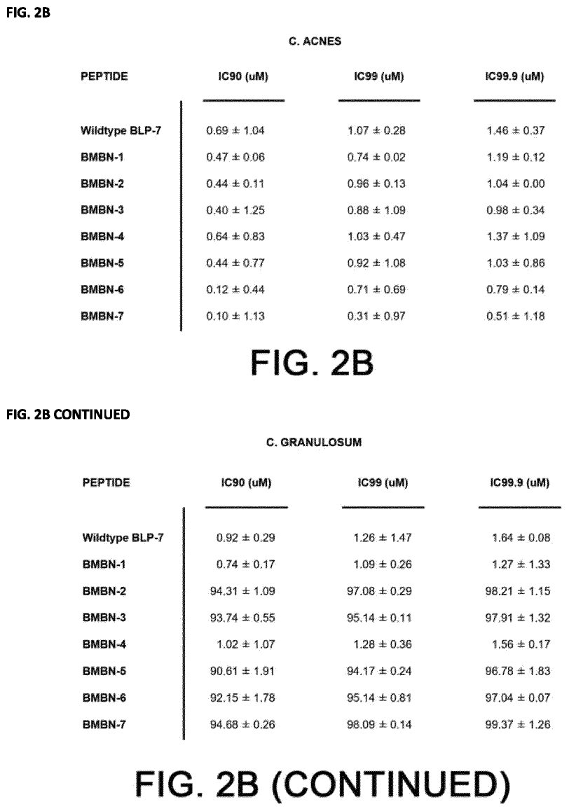

FIG. 2C: Comparison of the killing curves of wildtype BLP-7 and BMBN-7 on C. acnes (strain HL045PA1) represented by the amount of CFU (colony-forming units) of C. acnes per ml of culture medium by different concentrations of the peptides.

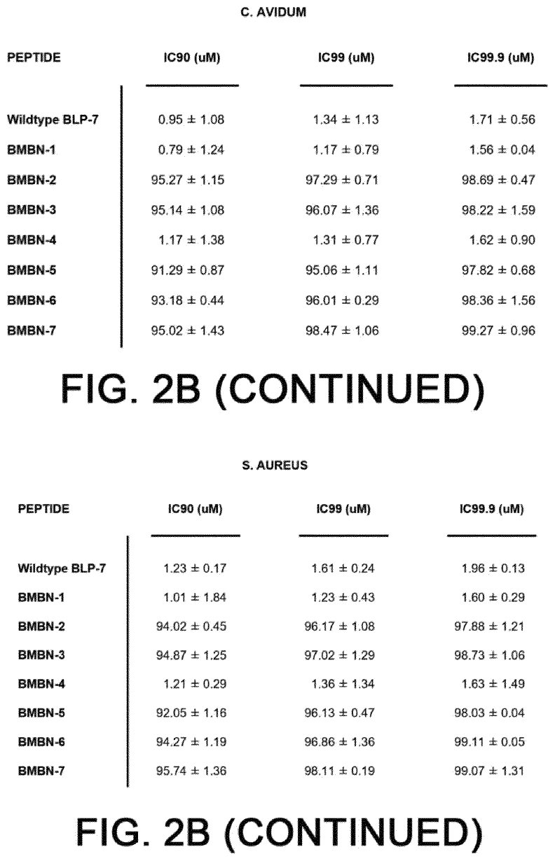

FIG. 3: Results of the Minimum Inhibitory Concentration (MIC) assay performed on C. acnes strains HL045PA1, RT6 HL110PA3, DSM 16379/KPA171202, and HL096PA3

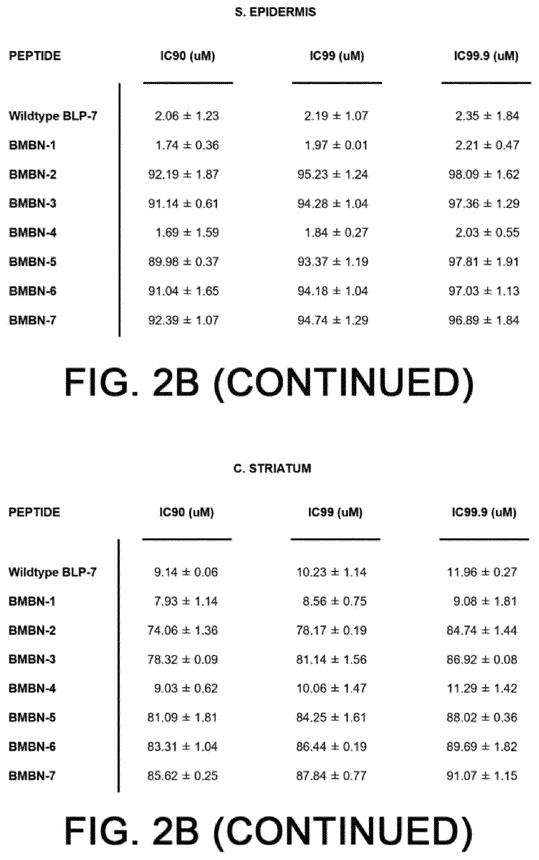

FIG. 4: Transmission Electron Microscopy imaging of untreated C. acnes (strain HL045PA1) and treated with BMBN-7. (arrow a) cell wall/membrane breakages, (arrow al) loss of cell wall, (arrow b) large cavity between cell wall and plasma membrane, (arrow c) leakage of intracellular contents, (arrow d) breakdown of cytoplasm into inclusion bodies, and (arrow e) loss of cytoplasm. Bar indicates 100 nm.

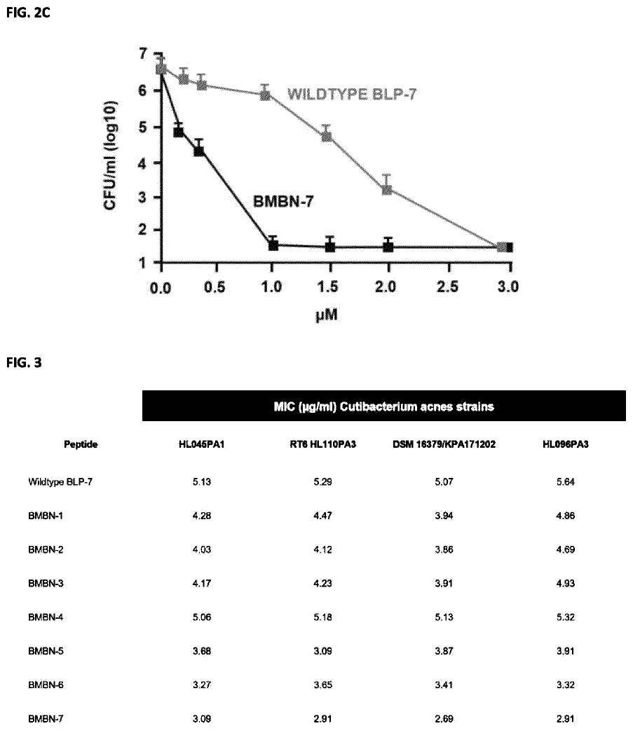

FIG. 5: Results of the hemolytic activity assay performed on human erythrocytes and peptides; wildtype BLP-7, BMBN-1, BMBN-2, BMBN-3, BMBN-4, BMBN-5, BMBN-6, BMBN-7.

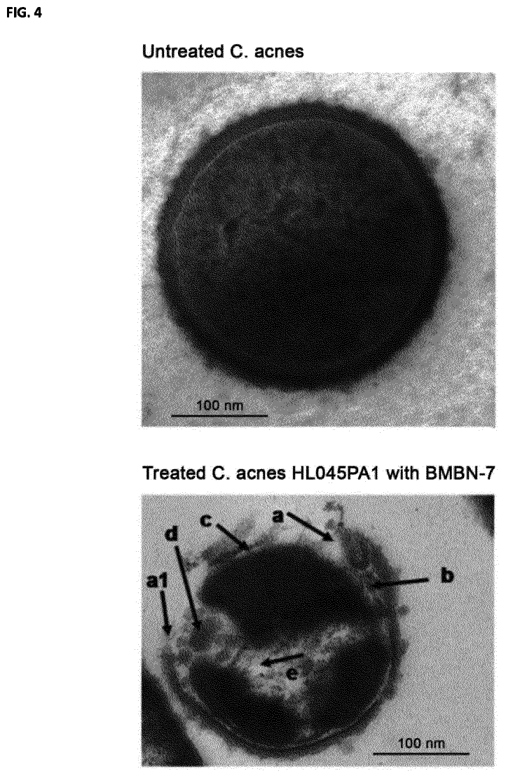

FIG. 6: Results of the cytotoxicity assay performed on an NL20 cell line and peptides; wildtype BLP-7, BMBN-1, BMBN-2, BMBN-3, BMBN-4, BMBN-5, BMBN-6, BMBN-7.

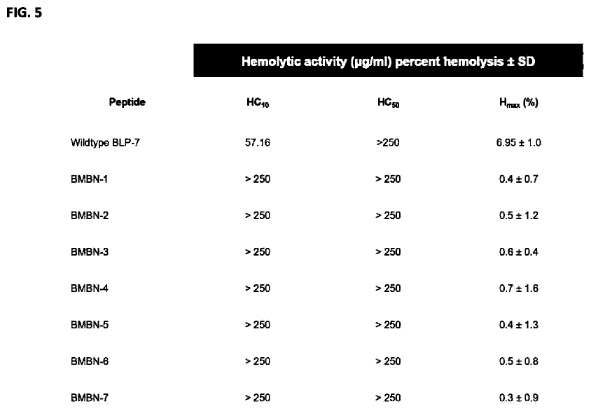

FIG. 7: Inhibition of inflammatory cytokines IL-8, IL-12, and TNF-α by wildtype BLP-7. Graphs A, C, E showing inhibition on protein level, graphs B, D, F showing inhibition on mRNA level.

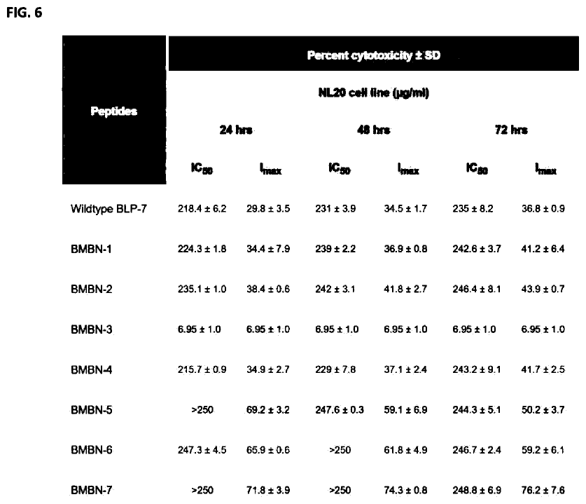

FIG. 8: Inhibition of inflammatory cytokines IL-8, IL-12, and TNF-α by BMBN-7. Graphs A, C, E showing inhibition on protein level, graphs B, D, F showing inhibition on mRNA level.

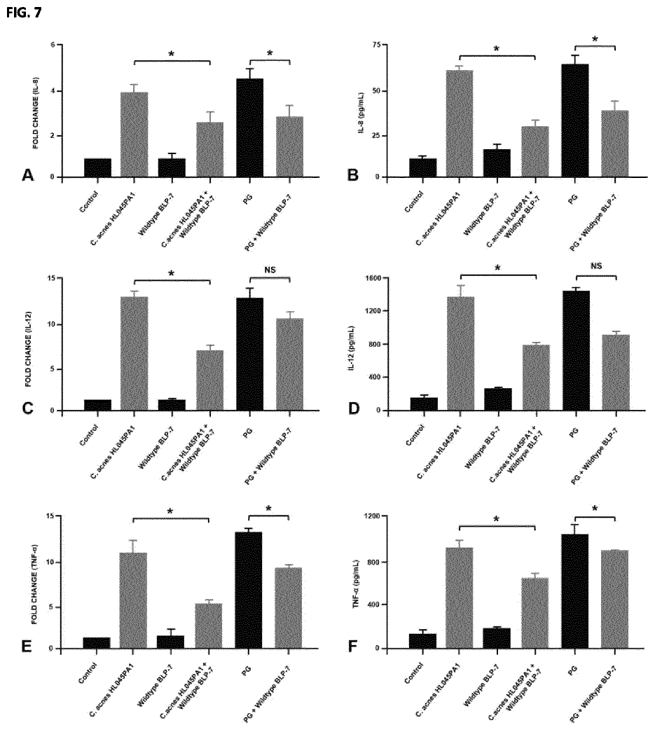

FIG. 9: Quantification of biofilm formation in 100 C. acnes clinical isolates. Cutibacterium granulosum DSM 20700 was used as a positive control. Experiments were performed in triplicate and each bar represents the mean±standard deviation.

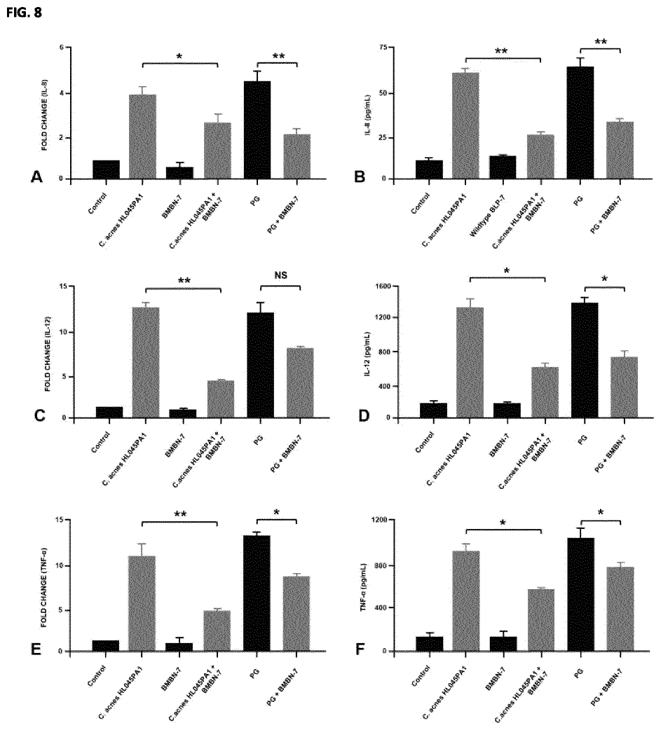

FIG. 10: The MICs, MBIC, MBEC, sample source and drug susceptibility of 100 C. acnes strains.

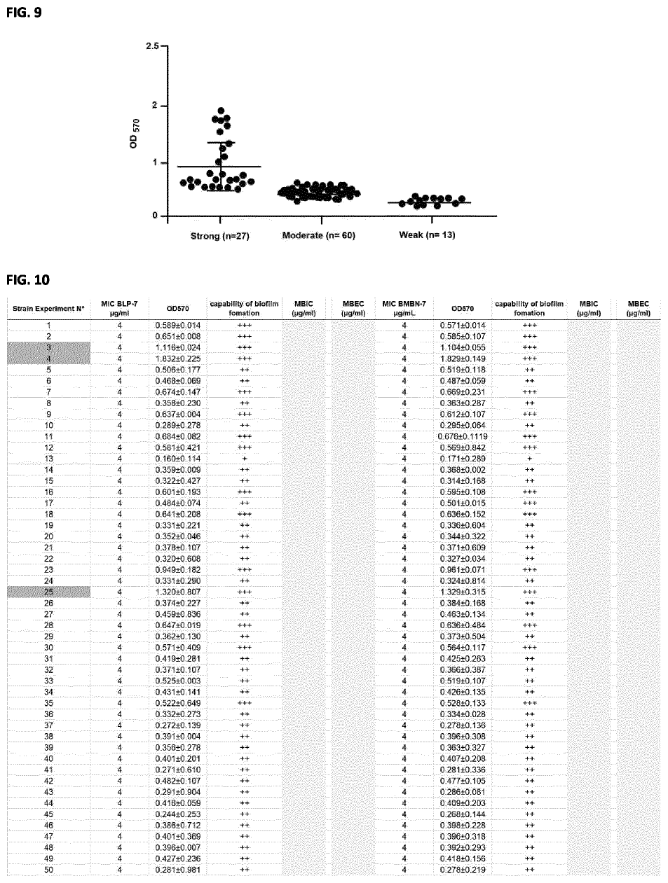

FIG. 11: The effects of wildtype BLP-7 and BMBN-7 on mature biofilms of C. acnes. The adherent biofilm was stained by crystal violet, and then the dye was extracted with ethanol, measured at a 570-nm absorbance, and presented as percentage of biofilm remains compared to untreated wells (0 μg/mL). All experiments were done in triplicate for statistical significance. * p<0.05; ** p<0.01.

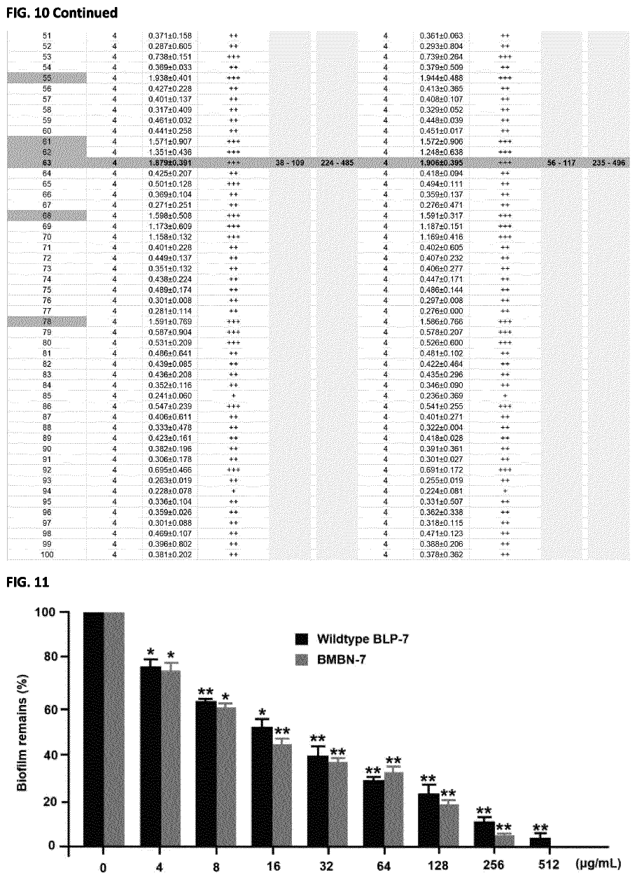

### REFERENCES

1. Epand R M, Vogel H J. Diversity of antimicrobial peptides and their mechanisms of action. Biochim Biophys Acta. 1999 Dec. 15; 1462(1-2):11-28.
2. Zasloff, M. Antimicrobial peptides of multicellular organisms. Nature 415, 389-395 (2002).
3. Lewies, A., Du Plessis, L. H. & Wentzel, J. F. Antimicrobial Peptides: the Achilles' Heel of Antibiotic Resistance? Probiotics & Antimicro. Prot. 11, 370-381 (2019).
4. Yu G, Baeder D Y, Regoes R R, Rolff J. Predicting drug resistance evolution: insights from antimicrobial peptides and antibiotics. Proc Biol Sci. 2018 Mar. 14; 285(1874):20172687.
5. Tomas Ganz, The Role of Antimicrobial Peptides in Innate Immunity, Integrative and Comparative Biology, Volume 43, Issue 2, April 2003, Pages 300-304.
6. Cederlund, A., Gudmundsson, G. H. and Agerberth, B. (2011), Antimicrobial peptides important in innate immunity. The FEBS Journal, 278: 3942-3951.
7. M. Marta Guarna, Richard Coulson, Evelina Rubinchik, Anti-inflammatory activity of cationic peptides: application to the treatment of acne vulgaris, FEMS Microbiology Letters, Volume 257, Issue 1, April 2006, Pages 1-6.
8. Yue Sun, Dejing Shang, “Inhibitory Effects of Antimicrobial Peptides on Lipopolysaccharide Induced Inflammation”, Mediators of Inflammation, vol. 2015, Article ID 167572, 8 pages, 2015.
9. Mangoni M L, McDermott A M, Zasloff M. Antimicrobial peptides and wound healing: biological and therapeutic considerations. Exp Dermatol. 2016 March; 25(3):167-73.
10. Raaymakers, C., Verbrugghe, E., Hernot, S. et al. Antimicrobial peptides in frog poisons constitute a molecular toxin delivery system against predators. Nat Commun 8, 1495 (2017).
11. Pretzel J, Mohring F, Rahlfs S, Becker K. Antiparasitic peptides. Adv Biochem Eng Biotechnol. 2013; 135:157-92.
12. Luis Rivas, Juan Román Luque-Ortega, David Andreu, Amphibian antimicrobial peptides and Protozoa: Lessons from parasites, Biochimica et Biophysica Acta (BBA)—Biomembranes, Volume 1788, Issue 8, 2009, Pages 1570-1581.
13. Deslouches B., Peter Di Y. Antimicrobial peptides with selective antitumor mechanisms: prospect for anticancer applications. Oncotarget. 2017; 8: 46635-46651.
14. Philip S. Stewart, Mechanisms of antibiotic resistance in bacterial biofilms, International Journal of Medical Microbiology, Volume 292, Issue 2, 2002, Pages 107-113, ISSN 1438-4221.
15. Sharma, D., Misba, L. & Khan, A. U. Antibiotics versus biofilm: an emerging battleground in microbial communities. Antimicrob Resist Infect Control 8, 76 (2019).
16. Walters M C 3rd, Roe F, Bugnicourt A, Franklin M J, Stewart P S. Contributions of antibiotic penetration, oxygen limitation, and low metabolic activity to tolerance of Pseudomonas aeruginosa biofilms to ciprofloxacin and tobramycin. Antimicrob Agents Chemother. 2003 January; 47(1):317-23.
17. Wright G D. Bacterial resistance to antibiotics: enzymatic degradation and modification. Adv Drug Deliv Rev. 2005 Jul. 29; 57(10):1451-70.
18. Davies J, Davies D. Origins and evolution of antibiotic resistance. Microbiol Mol Biol Rev. 2010 September; 74(3):417-33.
19. Hawkey P M. The origins and molecular basis of antibiotic resistance. BMJ. 1998 Sep. 5; 317(7159):657-60.
20. Wolf, A. J., Liu, G. Y. and Underhill, D. M. (2017), Inflammatory properties of antibiotic-treated bacteria. Journal of Leukocyte Biology, 101: 127-134.
21. Kristiansen, P. E., Fimland, G., Mantzilas, D. & Nissen-Meyer, J. Structure and mode of action of the membrane-permeabilizing antimicrobial peptide pheromone plantaricin A. J. Biol. Chem. 280, 22945-22950.
22. Asaduzzaman, S. M. & Sonomoto, K. Lantibiotics: diverse activities and unique modes of action. J. Biosci. Bioeng. 107, 475-487.
23. Breukink, E. et al. Use of the Cell Wall Precursor Lipid II by a Pore-Forming Peptide Antibiotic. Sci. 286, 2361.
24. Shai, Y. Mode of action of membrane active antimicrobial peptides. Biopolym. 66, 236-248.
25. Brogden, K. A. Antimicrobial peptides: Pore formers or metabolic inhibitors in bacteria? Nat. Rev. Microbiol. 3, 238-250.
26. Xi, D. et al. Design, expression and characterization of the hybrid antimicrobial peptide LHP7, connected by a flexible linker, against Staphylococcus and Streptococcus. Process. Biochem. 48, 453-461.
27. He, J. et al. Systematic Approach to Optimizing Specifically Targeted Antimicrobial Peptides against Streptococcus mutans. Antimicrob. Agents Ch.
28. He, J., Anderson, M. H., Shi, W. & Eckert, R. Design and activity of a ‘dual-targeted’ antimicrobial peptide. Int. J. Antimicrob. Ag. 33, 532-537.
29. Eckert, R. et al. Adding selectivity to antimicrobial peptides: Rational design of a multidomain peptide against Pseudomonas spp. Antimicrob. Agents Ch. 50, 1480-1488.
30. Eckert, R. et al. Targeted Killing of Streptococcus mutans by a Pheromone-Guided “Smart” Antimicrobial Peptide. Antimicrob. Agents Ch. 50, 3651-3657.
31. Qiu, X. Q. et al. An engineered multidomain bactericidal peptide as a model for targeted antibiotics against specific bacteria. Nat. Biotechnol. 21.

32 Solid Phase Peptide Synthesis. I. The Synthesis of a Tetrapeptide R. B. Merrifield. Journal of the American Chemical Society 1963 85 (14), 2149-2154.
33. Stewart, John Morrow, 1924-Solid phase peptide synthesis. Rockford, Ill.: Pierce Chemical Co., 1984.
34. Robert D. Walkup, Peptides: Synthesis, Structures, and Applications Edited by Bernd Gutte (Biochemisches Institut der Universit Zurich). Academic Press, San Diego, C A. 1995. Journal of Natural Products 1996 59 (12), 1219-1220.
35. Sambrook, J., Fritsch, E. R., & Maniatis, T. (1989). Molecular Cloning: A Laboratory Manual (2nd ed.). Cold Spring Harbor, NY: Cold Spring Harbor Laboratory Press.
36. Silhavy, T. J., Brennan, M. L. and Enquist, L. A. W., Experiments with Gene Fusions, Cold Spring Harbor Laboratory Cold Press Spring Harbor, N.Y. (1984); Ausubel, F. M. et al., Current Protocols in Molecular Biology, published by Greene Publishing Assoc. and Wiley-Interscience (1987)
37. Paul Lloyd-Williams, Fernando Albericio, Ernest Giralt, Chemical Approaches to the Synthesis of Peptides and Proteins.
38. Miklos Bodanszky, Agnes Bodanszky, The Practice of Peptide Synthesis.
39. Lars Andersson Lennart Blomberg Martin Flegel Ludek Lepsa Bo Nilsson Michael Verlander, Large-scale synthesis of peptides, Biopolymers (Peptide Science), (2000) 55:227-250.
40. Thomas D. Brock in Biotechnology: A Textbook of Industrial Microbiology, Second Edition (1989) Sinauer Associates, Inc., Sunderland, Mass., or Deshpande, Mukund V., Appl. Biochem. Biotechnol., 36, 227, (1992).
41. Bellion et al., Microb. Growth C1 Compd., [Int. Symp.], 7th (1993), 415-32. Editor(s): Murrell, J. Collin; Kelly, Don P. Publisher: Intercept, Andover, UK.
42. Richard F. Taylor, Protein immobilization: fundamentals and applications, publisher: New York, M. Dekker, 1991.
43. Zelezetsky, I., Pag, U., Sahl, H. G. & Tossi, A. Tuning the biological properties of amphipathic alpha-helical antimicrobial peptides: rational use of minimal amino acid substitutions. Peptides 26, 2368-2376 (2005).
44. Lee, S. H., Jaganath, I. B., Wang, S. M. & Sekaran, S. D. Antimetastatic effects of Phyllanthus on human lung (A549) and breast (MCF-7) cancer cell lines. PLOS One 6, e20994 (2011).
45. Alyu, F.; Dikmen, M. Inflammatory aspects of epileptogenesis: Contribution of molecular inflammatory mechanisms. Acta Neuropsychiatr. 2017, 1, 1-16.
46. Demina, Olga et al. “Role of Cytokines in the Pathogenesis of Acne.” International Journal of Biomedicine 7 (2017): 37-40.
47. O'Toole G A. Microtiter dish biofilm formation assay. J Vis Exp. 2011 Jan. 30; (47):2437.
48. Badmasti F, Siadat S D, Bouzari S, Ajdary S, Shahcheraghi F. Molecular detection of genes related to biofilm formation in multidrug-resistant Acinetobacter baumannii isolated from clinical settings. J Med Microbiol.
49. Abouelhassan, Y., Yang, Q., Yousaf, H., Nguyen, M. T., Rolfe, M., Schultz, G. S., et al. (2017). Nitroxoline: a broad-spectrum biofilm-eradicating agent against pathogenic bacteria. Int. J. Antimicrob. Agents 49, 247-251.

1-15. (canceled)

16. A polypeptide comprising an amino acid sequence with at least 97% sequence homology to:
(SEQ ID NO: 9)
GIGGALLSAG X¹SALX²GLAX³G LAEHFAN
wherein:
X¹, X², and X³ independently represent K (lysine); E (glutamic acid), or Q (glutamine);
at least one of X¹, X² and X³ is independently selected from E (glutamic acid) or Q (glutamine); and
the polypeptide comprises at least 35 amino acids.

17. The polypeptide according to claim 16, wherein one or more of the amino acids are D-amino acids.

18. The polypeptide according to claim 16, wherein one or more of the amino acids are L-amino acids.

19. The polypeptide according to claim 16, wherein the N-terminal is at amino acid 1 of the amino acid sequence.

20. The polypeptide according to claim 16, wherein the C-terminal is at amino acid 1 of the amino acid sequence.

21. The polypeptide according to claim 20, wherein the polypeptide comprises D-amino acids.

22. The polypeptide according to claim 16, wherein at least two of X¹, X², and X³ are independently selected from E (glutamic acid), or Q (glutamine).

23. The polypeptide according to claim 16, wherein the polypeptide is N-terminally modified, C-terminally modified, or both.

24. The polypeptide according to claim 23, wherein the polypeptide is N-terminally modified, and wherein the modification is selected from an acetyl residue, a hexanoyl residue, a decanoyl residue, a myristoyl residue, a NH—(CH₂—CH₂—O)₁₁—CO— residue, or a propionyl residue.

25. The polypeptide according to claim 23, wherein the polypeptide is C-terminally modified, and wherein the modification comprises an amide-, NH—(CH₂—CH₂—O)₁₁—CO-amide-, or one or two amino-hexanoyl groups.

26. The polypeptide according to claim 16, further comprising a C. acnes specific quorum sensing motif having a sequence selected from ERNNF, ERNNT, or ERNNY.

27. The polypeptide according to claim 16, wherein the polypeptide has an amino acid sequence with at least 93% sequence homology to SEQ ID NO:8.

28. A pharmaceutical composition comprising:
the polypeptide according to claim 16; and
at least one pharmaceutically acceptable carrier, diluent, and/or excipient.

29. The pharmaceutical composition according to claim 28, wherein the pharmaceutical composition is formulated for topical administration.

30. The pharmaceutical composition according to claim 28, wherein the pharmaceutical composition is formulated as a crème, a gel, an ointment, a lotion, a foam, a suspension, a spray, an aerosol, or a powder aerosol.

31. The pharmaceutical composition according to claim 28, further comprising an antimicrobial agent.

32. The pharmaceutical composition according to claim 28, further comprising a controlled release carrier and/or a targeted delivery carrier.

33. A method of treating a microbial infection, comprising:
administering a therapeutically effective amount of the pharmaceutical composition according to claim 28 to a subject in need thereof.

34. The method according to claim 33, wherein the microbial infection is selected from a Cutibacterium acnes infection, a Staphylococcus aureus infection, a Methicillin-Resistant Staphylococcus aureus (MRSA) infection, or a Gardnerella Vaginalis infection.

35. The method according to claim 33, wherein the microbial infection is a Cutibacterium acnes infection.


SEQUENCE LISTING (9 sequences)

SEQ ID NO: 1 - protein, 27 aa, Propionibacterium acnes
GIGGALLSAGKSALKGLAKGLAEHFAN

SEQ ID NO: 2 - protein, 27 aa, Propionibacterium acnes
  LIPID (1)..(1): PALMITATE
  MOD_RES (27)..(27): AMIDATION
GIGGALLSAGKSALKGLAKGLAEHFAN

SEQ ID NO: 3 - protein, 35 aa, Propionibacterium acnes
  LIPID (1)..(1): PALMITATE
  MOD_RES (35)..(35): AMIDATION
ERNNFGGGGIGGALLSAGKSALKGLAKGLAEHFAN

SEQ ID NO: 4 - protein, 35 aa, Propionibacterium acnes; identical to SEQ ID NO: 3
  LIPID (1)..(1): PALMITATE
  MISC_FEATURE (1)..(35): D-amino acids, the chiral form of L-amino acids
  MOD_RES (35)..(35): AMIDATION

SEQ ID NO: 5 - protein, 35 aa, Propionibacterium acnes
  LIPID (1)..(1): PALMITATE
  MOD_RES (35)..(35): AMIDATION
EFGGQGGGGIGGALLSAGKSALKGLAKGLAEHFAN

SEQ ID NO: 6 - protein, 35 aa, Propionibacterium acnes
  LIPID (1)..(1): PALMITATE
  MOD_RES (35)..(35): AMIDATION
ERNNFGGGGIGGALLSAGESALKGLAKGLAEHFAN

SEQ ID NO: 7 - protein, 35 aa, Propionibacterium acnes
  LIPID (1)..(1): PALMITATE
  MISC_FEATURE (1)..(35): retro-inverso sequence
  MOD_RES (35)..(35): AMIDATION
ERNNFGGGNAFHEALGKALGKLASEGASLLAGGIG

SEQ ID NO: 8 - protein, 35 aa, Propionibacterium acnes; identical to SEQ ID NO: 6
  MISC_FEATURE (1)..(35): D-amino acids, the chiral form of L-amino acids
  LIPID (1)..(1): PALMITATE
  MOD_RES (35)..(35): AMIDATION

SEQ ID NO: 9 - protein, 27 aa, Artificial Sequence
  Xaa stands for K, E or Q
  VARIANT (11)..(11): Xaa is K, E, or Q
  VARIANT (15)..(15): Xaa is K, E, or Q
  VARIANT (19)..(19): Xaa is K, E, or Q
GIGGALLSAGXSALXGLAXGLAEHFAN